(19) United States
(12) Patent Application Publication
(10) Pub. No.: US 2021/0261401 A1
(43) Pub. Date: Aug. 26, 2021

(54) AUTOMATED OIL DIFFUSER

(71) Applicant: Healixir Health, LLC, Salt Lake City, UT (US)

(72) Inventors: David Aiderzada, Salt Lake City, UT (US); Cameron Hendricks, Salt Lake City, UT (US)

(73) Assignee: Healixir Health, LLC, Salt Lake City, UT (US)

(21) Appl. No.: 17/183,633

(22) Filed: Feb. 24, 2021

Related U.S. Application Data
(60) Provisional application No. 62/980,651, filed on Feb. 24, 2020.

(51) Int. Cl. B67D 1/14; B67D 1/00

(52) U.S. Cl. CPC B67D 1/1472; B67D 2001/1483; B67D 1/0085; B67D 1/0019

(57) ABSTRACT

A valve for dispensing metered volumes of fluid includes a valve body configured to interface with a container holding a fluid. The valve body defines a fluid lumen that extends along at least a portion of the length of the valve body. A valve tip is in communication with the fluid lumen. An actuator is positioned within the valve body. A plunger is positioned within the valve body and in communication with the fluid lumen. The plunger is moveable by the actuator to open the fluid lumen. The plunger is biased in a closed direction. The valve includes a valve cap and a controller that is configured to activate the actuator to dispense a volume of fluid from the valve.

### CROSS-REFERENCES TO RELATED APPLICATIONS

This application claims the benefit of U.S. Provisional Application No. 62/980,651, entitled “AUTOMATED OIL DIFFUSER,” filed on Feb. 24 2020, the entire contents of which is hereby incorporated by reference.

### BACKGROUND OF THE INVENTION

Conventional essential oil diffusers typically require manual mixing of blends of oils directly into the base of a diffuser. This requires more work on the part of the user and may make it more difficult to precisely mix various blends, as small volumes of oils may be difficult to measure manually. Additionally, as manual mixing is required, automation of mixing and diffusing blends of oils are not possible. Improvements in the area of dispensing, mixing, and diffusing oil blends are desired.

### BRIEF SUMMARY OF THE INVENTION

In one embodiment, a valve for dispensing metered volumes of fluid is provided. The valve may include a valve body configured to be interfaced with a container holding a fluid. The valve body may define a fluid lumen that extends along at least a portion of the length of the valve body. A valve tip may be in communication with the fluid lumen. The valve may include an actuator positioned within the valve body. The valve may include a plunger positioned within the valve body and in communication with the fluid lumen. the plunger may be moveable by the actuator to open and close the fluid lumen. The plunger may be biased in a closed direction. The valve may include a controller that is configured to activate the actuator to dispense a volume of fluid from the valve.

In some embodiments, the valve may further include a valve cap. The controller may be disposed in one or both of the valve body or the valve cap. The valve cap may include a magnetic element that is used to orient the valve cap when inserted into an external device. The valve cap may funnel fluid from the fluid channel through a tip of the valve cap. Operation of the actuator may be driven by one or both of a volume of fluid to be dispensed and a viscosity of the fluid. The valve may include a fluid level sensor that is configured to detect a presence of the fluid within the container. The fluid level sensor may include a capacitive sensor that is coupled with the valve body. The controller may be further configured to determine a total volume of the fluid within the container base on a signal received from the fluid level sensor.

In another embodiment, a fluid dispensing assembly is provided. The assembly may include a container storing a volume of fluid. The container may include a neck. The assembly may include a valve. The valve may include a valve body configured to interface with the container. The valve body may define a fluid lumen that extends along at least a portion of the length of the valve body. A valve tip may be in communication with the fluid lumen. The valve may include an actuator positioned within the valve body. The valve may include a plunger positioned within the valve body and in communication with the fluid lumen. The plunger may be moveable by the actuator to open and close the fluid lumen. The plunger may be biased in a closed direction. The valve may include a controller that is configured to activate the actuator to dispense a metered amount of fluid from the container via the valve.

In some embodiments, the plunger may be biased in the closed direction by a spring element. The actuator may include a solenoid. The plunger may include a ferromagnetic material and is actuated by current passing through the solenoid. The valve body may further define at least one air return channel. The fluid lumen may be sealed off from electronic components of the valve. The assembly may include a Euro dropper disposed between a storage region of the container and the valve. The valve may further include an electrical connector disposed on an outer surface of the valve. The electrical connector may be coupleable to one or both of a power source and an external processor. The valve body may define a recess that receives at least a portion of the neck of the container and that secures the valve with the container. The fluid may include an essential oil. The fluid may include a multivitamin.

In another embodiment, a method of dispensing a fluid is provided. The method may include receiving, at a processor of a valve, a signal to dispense a metered volume of a fluid from a container that is interfaced with the valve. The method may include triggering an actuator of the valve to move a plunger to an open position to dispense the metered volume of the fluid from the container. One or both of a duration and a distance of movement of the plunger to the open position may be based at least in part on one or both of an amount of the metered volume and a viscosity of the fluid.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a set of parentheses containing a second label that distinguishes among the similar components. If only the first reference label is used in the specification, the description is applicable to any one of the similar components having the same first reference label irrespective of the second reference label.

FIG. 1A illustrates an isometric view of a diffuser according to embodiments of the invention.

FIG. 1B illustrates a partially exploded view of the diffuser of FIG. 1A.

FIG. 1C illustrates a partially exploded view of the diffuser of FIG. 1A.

FIG. 1D illustrates a cross-sectional view of the diffuser of FIG. 1A.

FIG. 2A illustrates an isometric view of a diffuser according to embodiments of the invention.

FIG. 2B illustrates a side elevation view of the diffuser of FIG. 2A.

FIG. 2C illustrates a front elevation view of the diffuser of FIG. 2A.

FIG. 2D illustrates an isometric view of the diffuser of FIG. 2A.

FIG. 2E illustrates a top plan view of the diffuser of FIG. 2A.

FIG. 2F illustrates a side elevation cross-sectional view of the diffuser of FIG. 2A.

FIG. 2G illustrates a bottom isometric view of the diffuser of FIG. 2A.

FIG. 2H illustrates a side elevation view of the diffuser of FIG. 2A with a bottle positioned to receive a dispensed fluid according to embodiments.

FIG. 3A is an isometric view of a diffuser according to embodiments of the invention.

FIG. 3B is a partially disassembled isometric view of the diffuser of FIG. 3A.

FIG. 3C is a partially disassembled isometric view of the diffuser of FIG. 3A.

FIG. 3D is a partially disassembled top plan view of the diffuser of FIG. 3A.

FIG. 3E is a partially disassembled isometric view of the diffuser of FIG. 3A.

FIG. 3F is a partially disassembled top plan view of the diffuser of FIG. 3A.

FIG. 4A illustrates an isometric view of a valve according to embodiments of the invention.

FIG. 4B illustrates a cross-sectional view of the valve of FIG. 4A.

FIG. 4C illustrates an exploded view of the valve of FIG. 4A.

FIG. 5A illustrates an isometric view of a valve according to embodiments of the invention.

FIG. 5B illustrates an exploded view of the valve of FIG. 5A.

FIG. 5C illustrates a front elevation view of the valve of FIG. 5A.

FIG. 5D illustrates the valve of FIG. 5A disposed within a container according to embodiments of the invention.

FIG. 5E illustrates the valve of FIG. 5A disposed within a container according to embodiments of the invention.

FIG. 6A illustrates an isometric view of a valve according to embodiments of the invention.

FIG. 6B illustrates an exploded view of the valve of FIG. 6A.

FIG. 6C illustrates the valve of FIG. 6A disposed within a container according to embodiments of the invention.

FIG. 7A illustrates an exploded cross-sectional view of a valve according to embodiments of the present invention.

FIG. 7B illustrates an exploded cross-sectional view of the valve of FIG. 7A.

FIG. 7C illustrates a cross-sectional view of the valve of FIG. 7A.

FIG. 7D illustrates a cross-sectional view of the valve of FIG. 7A.

FIG. 8 is a flowchart illustrating a process for diffusing a fluid according to embodiments of the invention.

FIGS. 9A-9F illustrate a user interface for a mobile application for controlling a diffuser according to embodiments of the invention.

FIG. 10 is a schematic illustrating a computing device according to embodiments of the invention.

### DETAILED DESCRIPTION OF THE INVENTION

The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Embodiments of the present invention are directed to systems and methods for providing selectable and/or customizable scents to be mixed and dispensed using a single device, as well as dispensing mechanisms (such as valves) that enable precise automated dispensing of oils and other fluids. Such systems enable users to create and diffuse any number of combinations of scent and essential oil mixtures with a press of a button. Embodiments may provide systems in which oils may be mixed and used immediately and/or systems in which oils are mixed for later diffusion, topical application, and/or other use. Additionally, embodiments enable a user to control the operation of the mixing/diffusing device remotely and/or to have a scent mixed and/or diffused at a particular time of day. While discussed primarily in relation to essential oils, it will be appreciated that the present invention is not so limited and may be used in numerous other applications in which substances need to be dispensed, mixed, atomized, diffused, and/or otherwise produced. For example, embodiments of the present invention may encompass valves and/or other dispensing mechanisms that may be utilized in mixing drinks, flavoring agents, liquid multivitamins, supplements, and/or other liquids such as for adding scents, colors, etc., to base substances (such as a shampoo, conditioner, lotion, and/or other base substance). Additionally, it will be appreciated that as used herein, the terms oil and/or essential oil may be interpreted as including other liquid substances having desired color, aromatic, taste, health benefits, and/or other characteristics.

Embodiments relate to improvements in the control, organization, dispensing, and initiation of various blends or recipes (custom or known) of essential oils, carrier oils, and/or other fluids for uses related to health (aromatherapy, topical application, etc.), wellness (meditation, deep breathing, etc.), cleaning (detergents, soaps, etc.), cosmetics (perfumes, lotions, etc.), and/or various other uses. Embodiments of the present invention automate the process of dispensing, mixing, and diffusing blends of material and grants the user access to endless blends concocted by healthcare professionals and certified homeopaths and/or custom blends created by the user.

The blends of oils may be selected and the mixing of the blends may be initiated using a mobile application that can be accessed and triggered directly through touch and/or voice commands. The mobile application may operate in conjunction with a physical essential oil diffuser through a wireless connection.

In some embodiments, a process for operating an oil diffuser involves inserting essential oil (or other fluid) fluid dispensers into slots or ports formed within a diffuser. The diffuser and/or mobile application is initialized by inputting information associated with the oil within each fluid dispenser into the diffuser and/or mobile application. For example, if the oil present in slot or port 1 is peppermint oil, information regarding peppermint oil may be associated with port 1. This may include a name or other identifier of the oil, a viscosity of the oil, recipes associated with a given oil, a volume of the oil, and/or other information. In some embodiments, the information associated with the oil may be input by scanning (using the diffuser and/or mobile device) a visual identifier on the fluid dispenser that is encoded with the information, while in other embodiments, the information may be manually input or selected using the diffuser and/or mobile application. In yet other embodiments, the fluid dispenser itself may include a data chip that includes the oil information. This chip may be read by the diffuser, such as by using an RF antenna and/or direct electrical connection.

Once the fluid dispensers and oil information are input to the diffuser and/or mobile application, a database may be triggered that is populated with a list of oil blends or recipes that can be produced using the oils present in the diffuser. The user may select a pre-defined blend and/or a custom blend using the diffuser and/or the mobile application, which may then cause the diffuser to mix the selected blend of oils. In some embodiments, the database may be stored on the mobile device and/or the diffuser and may include a catalog of oil blend recipes. In other embodiments, the database may be stored on a remote server that may be accessed using a menu provided on the mobile application and/or the diffuser. In an aromatherapy scenario, the selection of the blend causes a signal to be sent to the diffuser that specifies water and oil proportions. Water is pumped from a tank into a mixing basin according to the amount specified in the blend. Valves of fluid dispensers that correspond to the oils that make up the blend may be opened to disperse a metered volume of each oil within the blend according to the particular proportion designated in the blend. The blend may be mixed and diffused for a period of time (which may be predetermined for each particular blend and/or selected by a user). In some embodiments, the diffuser and/or mobile application may also have the ability to control functions associated with the diffuser such as scent intensity, diffusion start/stop time, diffusion duration, etc.

In some embodiments, the recipes may be provided via machine learning, which uses huge datasets to determine if a user will like a certain recipe. For example, the device itself, a server, and/or a mobile application may leverage data received from the user to calculate suggested blends or suggested oils to add to the user's collection. In some embodiments, the machine learning may be based on criteria (such as, but not limited to, age, location, gender, previous oils used, current oils loaded, ethnicity, economic status, familial structure, exercise habits, weight/weight goals, the presence of animals in the user's environment, etc.) to determine what recipes a particular user may enjoy. In some embodiments, a user's demographic or other data may be compared to other users, with ingredients and/or recipes from similar users being suggested. This demographic and/or other data may be collected from various users via the mobile application. A backend server may use the data to learn which blends or oils a new user or current user may like. As more data is collected the algorithm may be refined to create something more specific for each user.

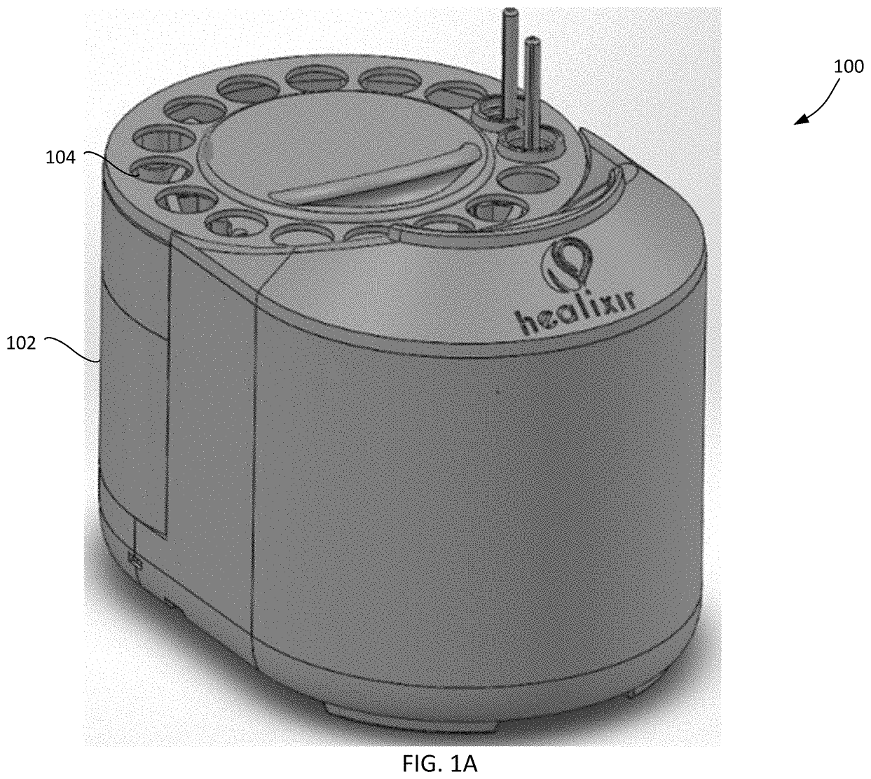

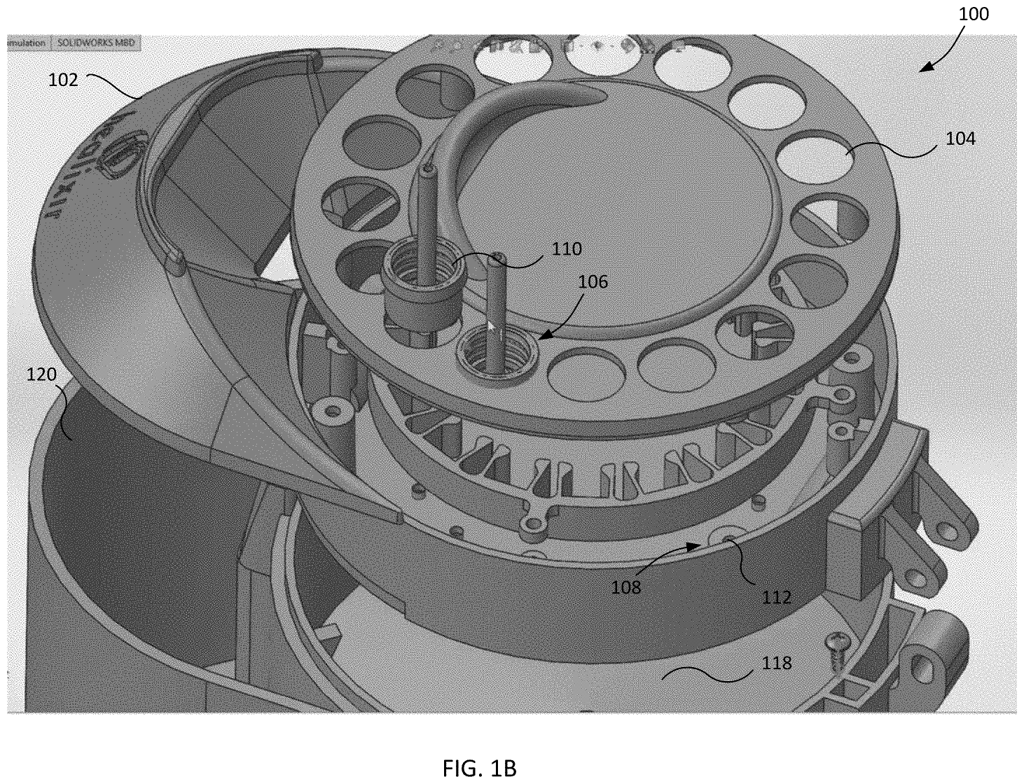

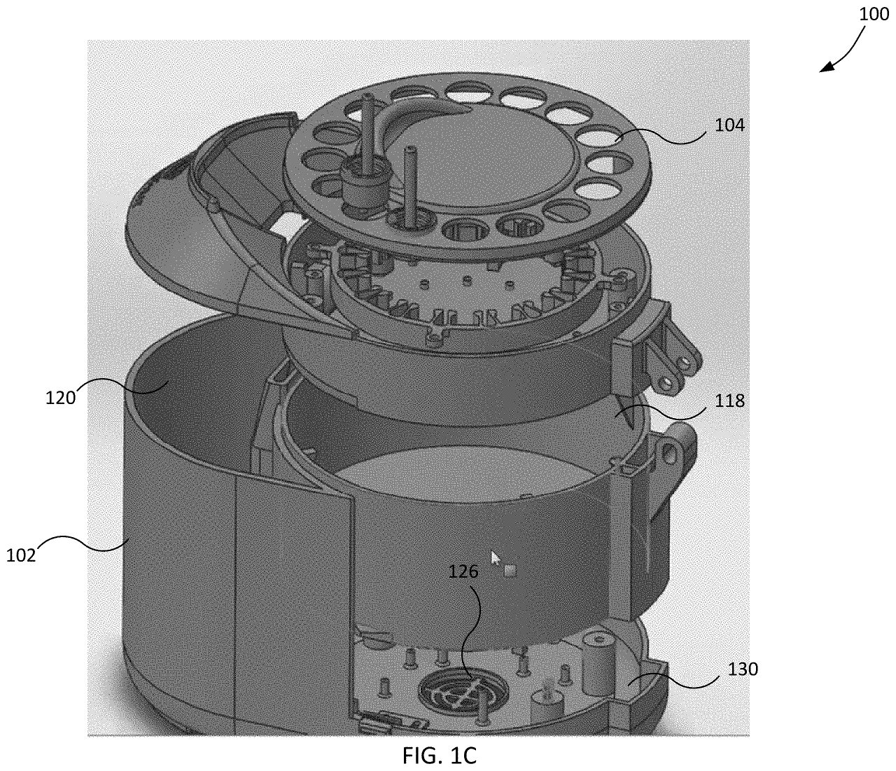

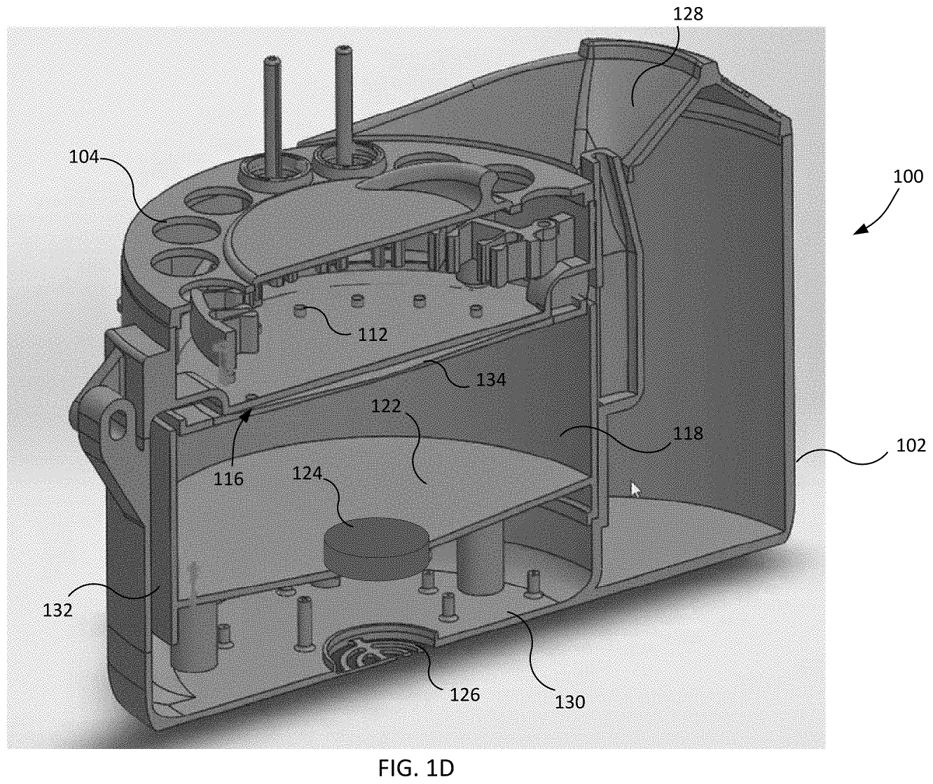

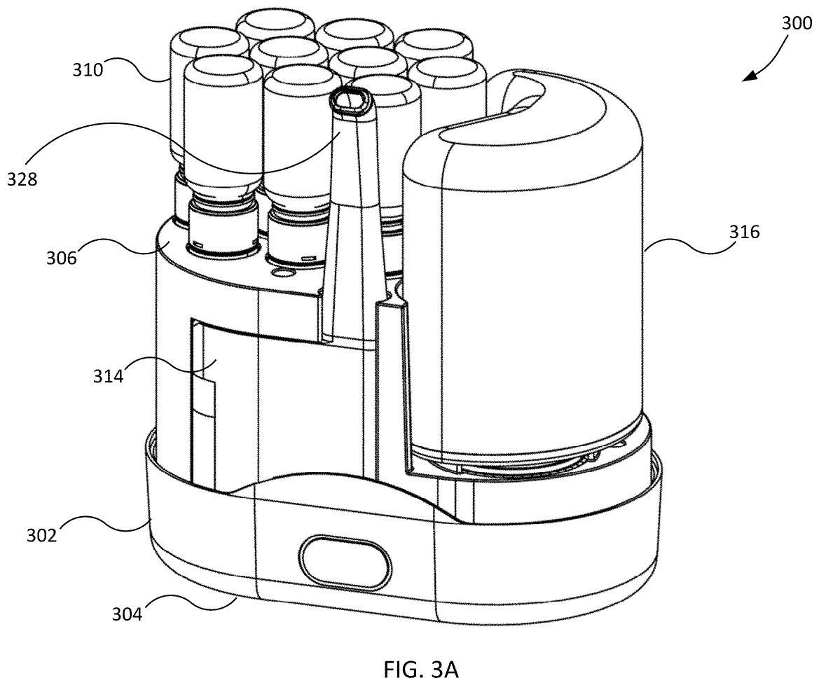

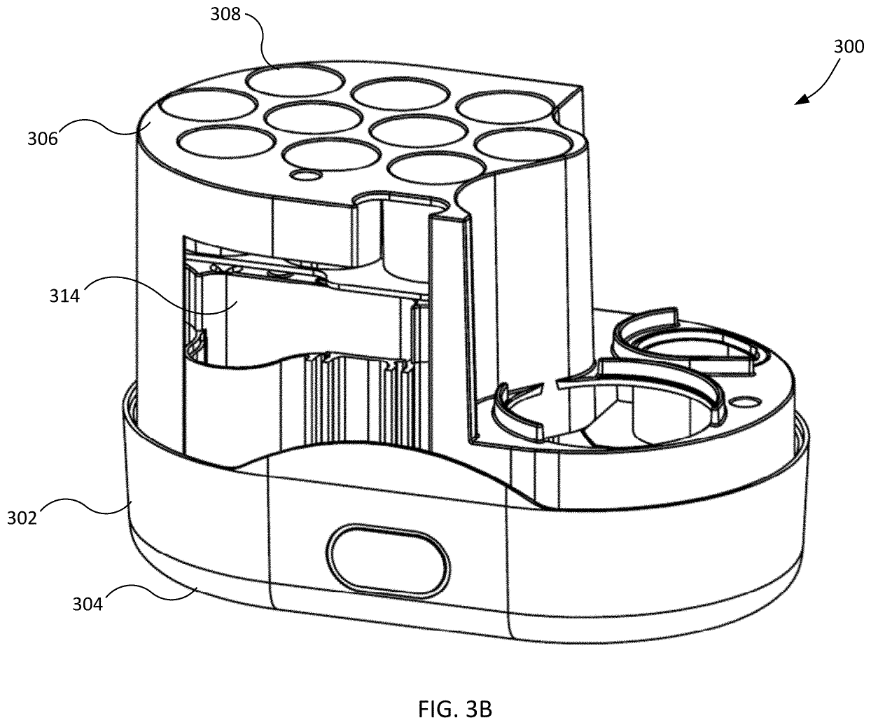

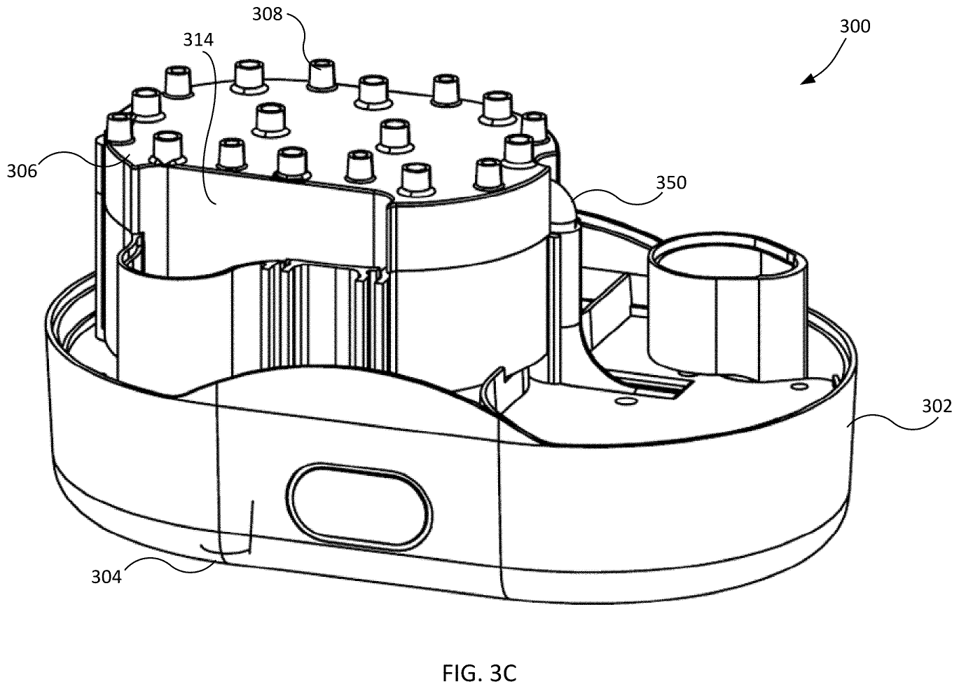

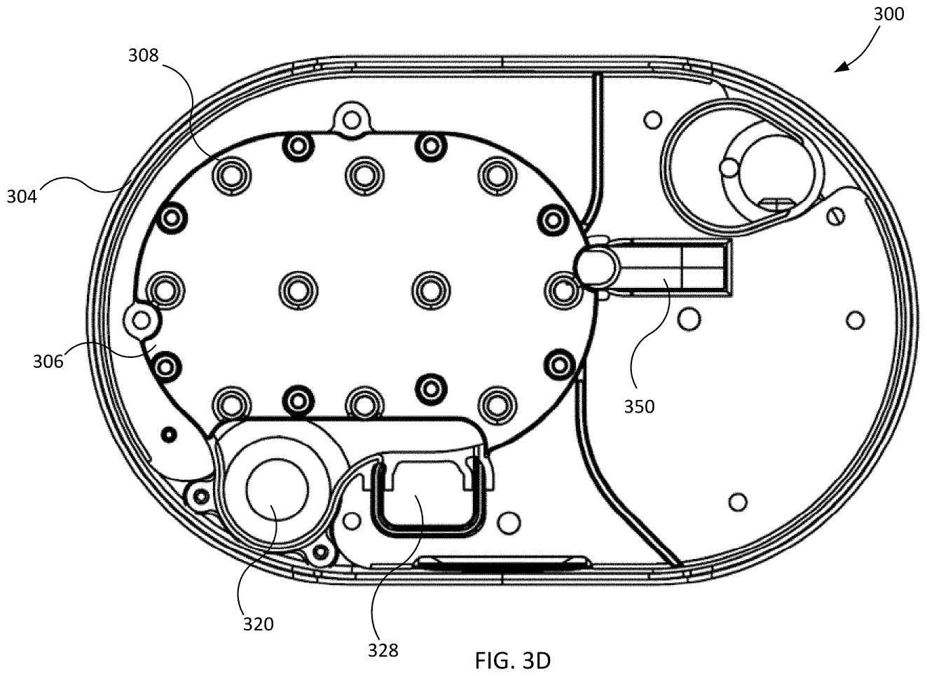

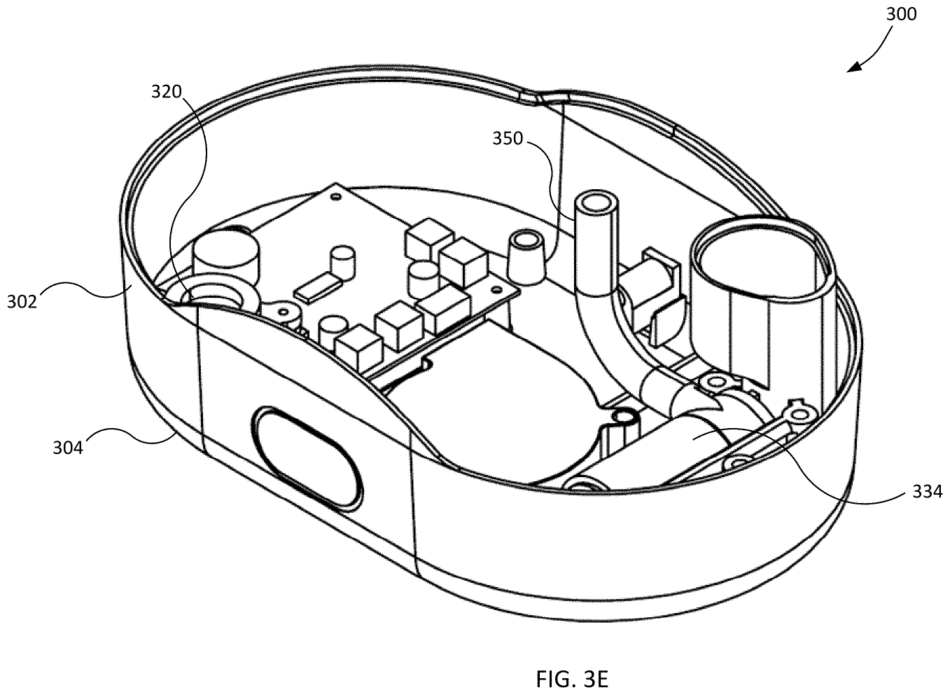

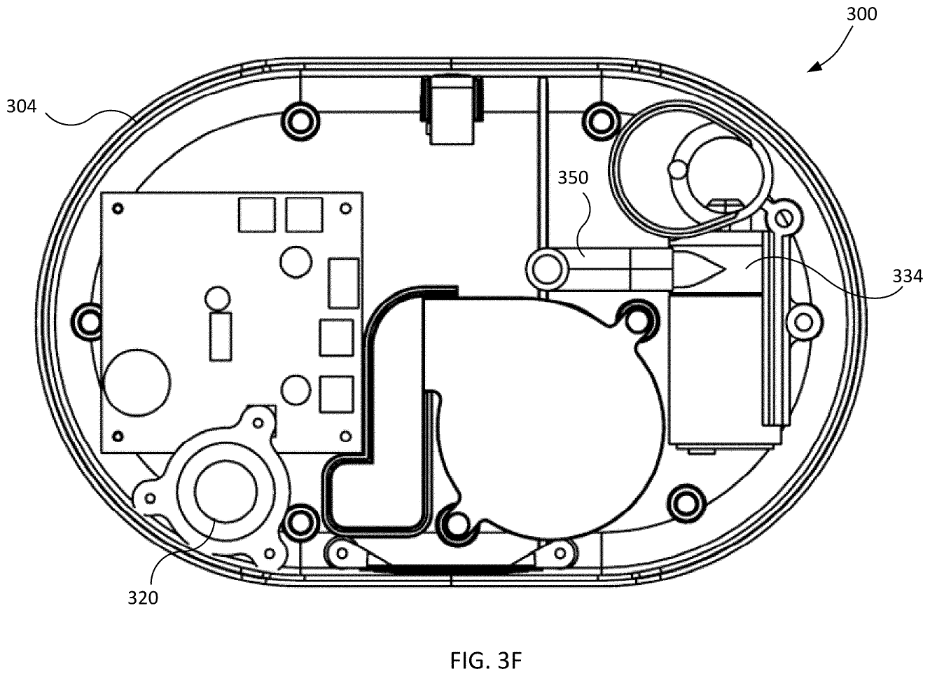

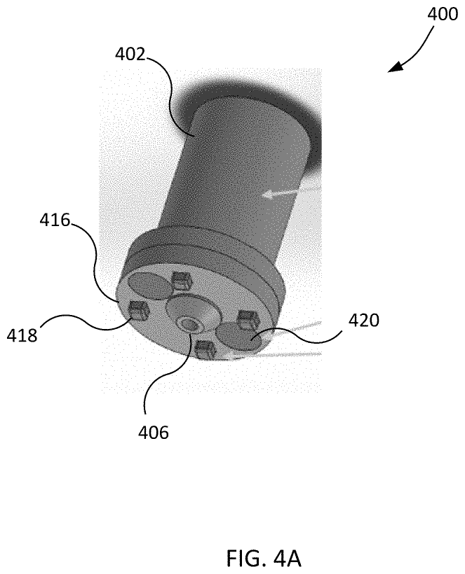

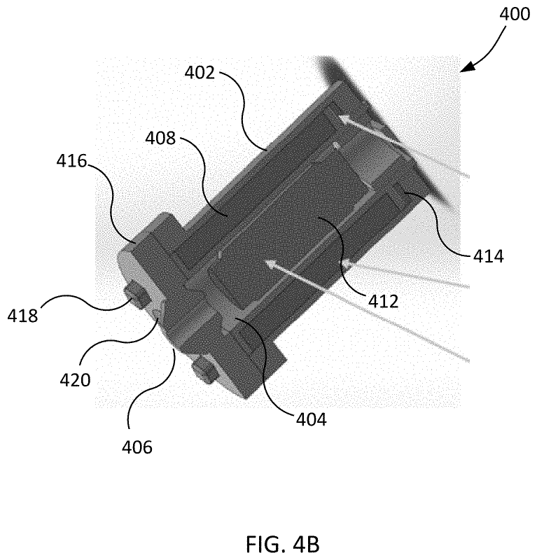

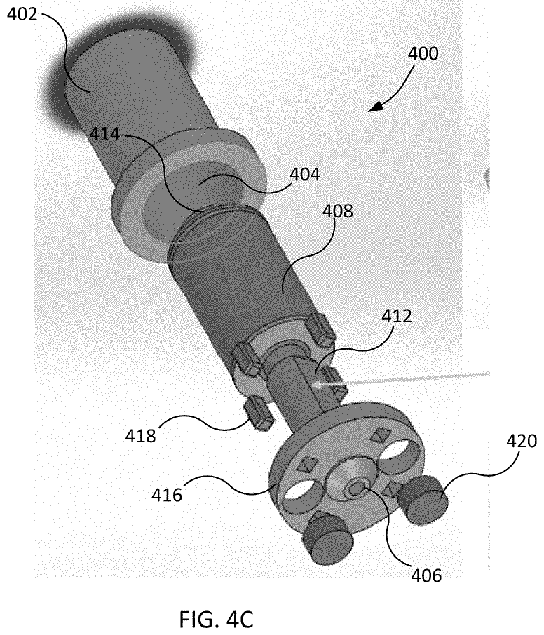

FIGS. 1A and 1B depict one embodiment of an oil diffuser 100. Oil diffuser 100 may include a housing 102. Housing 102 may define a number of fluid ports 104. Each fluid port 104 may include a first portion 106 that defines an opening through the exterior of the housing 102 through which a fluid dispenser (not shown; fluid dispensers often include a container and a dispensing valve, such as the valves described in relation to FIGS. 3A-4E) may be inserted. A tip of the fluid dispenser may be received by a second portion 108 of the fluid port 104. In some embodiments, the first portion 106 may include a connector that is used to secure a fluid dispenser within the first portion 106, such as a snap connector, magnet, and/or other coupling mechanism. As illustrated here, the first portion 106 may include a threaded connector 110 that allows a fluid dispenser to be screwed into the first portion 106 of the fluid port 104. In other embodiments, the fluid dispensers may be held within the fluid ports 104 using only gravity. While illustrated with fifteen fluid ports 104, it will be appreciated that any number of fluid ports 104 may be positioned on the oil diffuser 100, with greater numbers of fluid ports 104 being able to produce a greater variety of scents and/or other blends.

In some embodiments, each of the fluid ports 104 may include an electrical connector. Each electrical connector (not shown) may be configured to interface with a corresponding connector of a valve of a fluid dispenser received therein. In some embodiments, the electrical connector may be positioned on a side of the first portion 106 and/or formed in a side of the threaded connector 110 (or other coupling mechanism). In other embodiments, the electrical connector may be provided at or near a base of the second portion 108. Once interfaced with a corresponding connector of a fluid dispenser, the electrical connector may enable the oil diffuser 100 to provide power and/or instructions to the fluid dispenser that control the operation of a valve within the fluid dispenser. As just one example, a controller (not shown) of the oil diffuser 100 may send a signal that causes a valve of the fluid dispenser to open and/or close to dispense a metered volume of an essential oil stored within the fluid dispenser.

The second portion 108 may be configured to receive a volume of fluid from a tip of the fluid dispenser. The second portion 108 may define an aperture 112 that extends through the second portion 108 and a top surface 116 of a mixing basin 118. The mixing basin 118 may be positioned below the fluid ports 104 such that when oils are dispensed from the fluid dispensers, the oils flow through the aperture 112 and are drawn into the mixing basin 118 via gravity. The mixing basin 118 may hold a volume of water (or other fluid) in which the oils are dispensed to form a mixture. In some embodiments, the mixing basin 118 may be removably coupled with the housing 102 such that when a user wishes to add water to the mixing basin 118, the user may remove the mixing basin 118 from the housing 102. In other embodiments, the mixing basin 118 may be coupled with one or more fluid sources. For example, in some embodiments the oil diffuser 100 may include a water reservoir 120 that is in fluid communication with the mixing basin 118 such that a volume of water may be transferred from the water reservoir 120 to the mixing basin 118. In some embodiments, the volume of water may be supplied via one or more pumps and/or valves disposed within the housing 102. In other embodiments, gravity may be used to drain water from the water reservoir 120 to the mixing basin 118. The water reservoir 120 may include a cover that may be moved (such as by sliding and/or pivoting) and/or removed from the water reservoir 120 to allow the water reservoir 120 to be supplied with water from an external source. In some embodiments, the water reservoir 120 may be removably coupled with the housing 102 such that the water reservoir may be removed for easier filling and/or cleaning. In some embodiments, rather than, or in addition to, having a water reservoir 120, the oil diffuser 100 may include a water port that may be coupled directly with an incoming water line that may supply the mixing basin 118 and/or water reservoir 120 with a volume of water when a valve is actuated.

The mixing basin 118 may include a base plate 122 that supports the mixture of water and oils. In some embodiments, the base plate 122 may be sloped downward such that the mixture is drawn downward to a single area. For example, as illustrated here, the base plate 122 may be sloped toward a center of the base plate 122, although it will be appreciated that the base plate 122 may be sloped to direct the mixture to any desired area of the base plate 122. An atomizer 124 may be positioned at or near the lowest portion of the base plate 122 such that fluid is drawn toward the atomizer 124. The atomizer 124 may then receive at least a portion of the mixture of fluid and atomize the mixture into an atomized vapor or mist. The atomizer 124 may be an ultrasonic atomizer, a heat atomizer, evaporative atomizer, and/or a nebulizer.

For example, the atomizer 124 may be an ultrasonic atomizer that dispenses oils into the air as negative ions by using ultrasonic vibrations used to break down the mixture into micro particles, allowing the oil to be dispersed as a very fine mist into the air. This may be done, for example, by a disk or mesh being vibrated at a very high rate, for example, by using a piezoelectric and/or magnetostrictive actuator. While illustrated with a single atomizer 124, it will be appreciated that in some embodiments multiple atomizers (and possibly multiple types of atomizers) may be utilized, which may assist in atomizing the mixture at a greater rate.

The oil diffuser 100 may also include at least one fan 126 and/or other air flow device. The fan 126 is configured to push and/or otherwise draw the air containing the atomized mixture out of the air diffuser 100 via a ventilation port 128. As illustrated here, fan 126 is positioned in a separate ventilation chamber 130 positioned beneath the mixing basin 118, however, it will be appreciated that other arrangements are possible. For example, rather than being positioned in a separate ventilation chamber, the fan 126 may be positioned within the mixing basin 118 in some embodiments. In other embodiments, a ventilation chamber 130 may be used, but may not be positioned beneath the mixing basin 118. As just one example, the ventilation chamber 130 may be positioned to the side of the mixing basin 118. In the illustrated embodiment, the ventilation chamber 130 is in communication with the mixing basin 118 via a ventilation lumen 132. Ventilation lumen 132 may be defined by sidewalls and provides a flow path for air to be pushed or otherwise forced out of the ventilation chamber 130, through the mixing basin 118, and expelled through the ventilation port 128. The atomized particles of the mixture of oils and water may be entrained within the moving air and transported out of the diffuser 100. In some embodiments, the ventilation lumen 132 may be designed such that air is moved across a top of the mixing basin 118 to help entrain the atomized mixture within the airflow. As illustrated, the ventilation lumen 132 is disposed between along a sidewall of the mixing basin 118 and an outer wall of the housing 102 and connects a portion of the ventilation chamber 130 with a top portion of the mixing basin 118, however other designs are possible.

In some embodiments, the diffuser 100 may also include a removable mixing tray 134. Mixing tray 134 may be positionable below the fluid ports 104 within and/or above the mixing basin 118. For example, the mixing tray 134 may be positioned within a top portion of the mixing basin 118 such that the mixing tray is suspended above the base plate 122. The mixing tray may 134 be solid and may be used to collect mixtures of various oils dispensed via the fluid ports 104 prior to the oils being mixed with water in the mixing basin 118. This allows volumes of mixed recipes of oils to be dispensed, collected, and retained for topical treatments and/or later use. For example, once oil is deposited on the mixing tray 134, the mixing tray 134 may be removed from the diffuser 100, such as by sliding the mixing tray 134 out of a slot formed within the housing 102, and the oil mixture may be applied topically, poured into a container, and/or otherwise used outside of the diffuser 100. In some embodiments, a funnel that directs the oils into the mixing basin 118 and/or the mixing tray 134 may be coated with an oleo-phobic coating to increase amounts of oil that is delivered to the mixing basin 118 and/or the mixing tray 134.

The diffuser 100 may also include a controller (not shown) that is configured to control the operation of the fluid ports 104 (and fluid dispensers interfaced therewith), the atomizer 124, and the fan 126. For example, circuitry may be used to connect the controller with the various components. Additionally, as disclosed above, the controller may be coupled with the fluid dispensers via the electrical connectors, which allows the controller to actuate the valves of the various fluid dispensers. For example, actuation of valves of the various fluid dispensers may then dispense a volume of oil through one of the fluid ports. The controller may activate a particular set of valves based on a particular oil recipe. For example, an oil recipe may include directions for a particular volume for each of a number of oils must be dispensed and mixed to form a particular scent and/or other fluid characteristic. The volume may be identified in a specific volume and/or as relative parts (e.g. 1 part of a first oil, 2 parts of a second oil, etc.). As disclosed above, once the oils are dispensed according to the appropriate recipe, they are deposited in the mixing basin 118 and/or the removable mixing tray 134 for diffusion and/or other use. If the oils are dispensed into the mixing basin 118, the controller may then send commands to the atomizer 124 and/or fan 126 to diffuse the oils and expel the oils and water mixture into the air outside of the diffuser 100.

To properly dispense the correct types of oils, the controller may need to determine what kind of oil is present in each fluid dispenser disposed within each of the fluid ports 104. This may be done in several ways. For example, in some embodiments, a data chip within each fluid dispenser may be programmed during manufacture and/or by an end user (such as when the fluid dispenser is reusable and/or otherwise useable with different types of oils). The controller may read data from the data chip in one or more ways. For example, the data chip may be in communication with the controller via the electrical connector, allowing the controller to read information (such as a type of oil, viscosity of oil, etc.) from the data chip. In other embodiments, the data chip may be a radio frequency (RF) chip, such as an active or passive near field communication (NFC) chip. The controller may be coupled with an RF reader that is configured to produce an RF field that is modulated by the RF chip of the fluid dispensers to provide the information to the controller. In some embodiments, each RF chip may contain a small amount of data, such as an identifier of the bottle and/or an indexed value. The data may be read by the diffuser 100, which may then look up the identifier or other information in a local and/or cloud-based database. The diffuser 100 may then access various information about the contents of the dispenser, which may not be stored on the RD chip itself. For example, the diffuser 100 may be able to look up a type of fluid in the container, an amount of fluid within the container, characteristics associated with the fluid (such as viscosity), known recipes associated with the fluid, and/or other information.

In yet other embodiments, each fluid dispenser may include a barcode (or other computer readable identifier) that is encoded with information associated with a type of oil stored within the particular fluid dispenser. Prior to inserting each fluid dispenser into a respective one of the fluid ports 104, a user may scan the barcode to input the oil information (including an indicator of which port a given dispenser is being inserted) into the controller. In other embodiments, each of the fluid ports 104 may include a dedicated reader that scans the barcode and/or other computer readable identifier that is affixed to a fluid dispenser that is inserted within the fluid port 104. In some embodiments, the user may be able to program the controller with the oil information. For example, in some embodiments, the diffuser 100 may include a user interface, such as a touch screen, a keypad, and/or other input device that allows a user to input the information associated with the oil in a particular fluid dispenser. In other embodiments, a remote device, such as a mobile phone, tablet, e-reader, computer, and/or other computing device, may be used to input the oil information. For example, software, such as a mobile application, may be used to enable the remote device to interface with the controller via a communications interface of the diffuser 100.

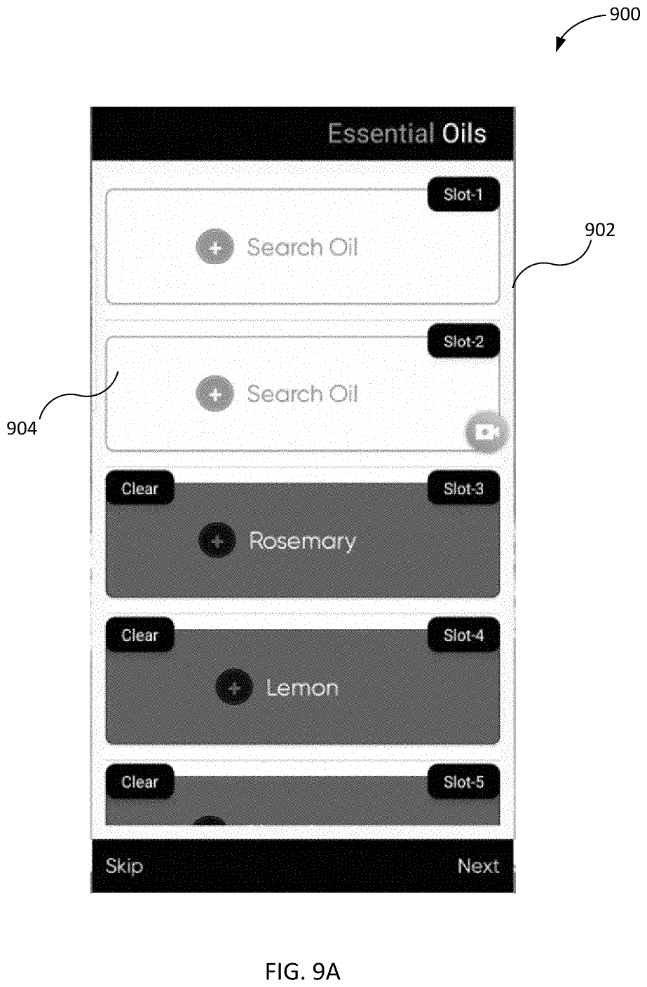

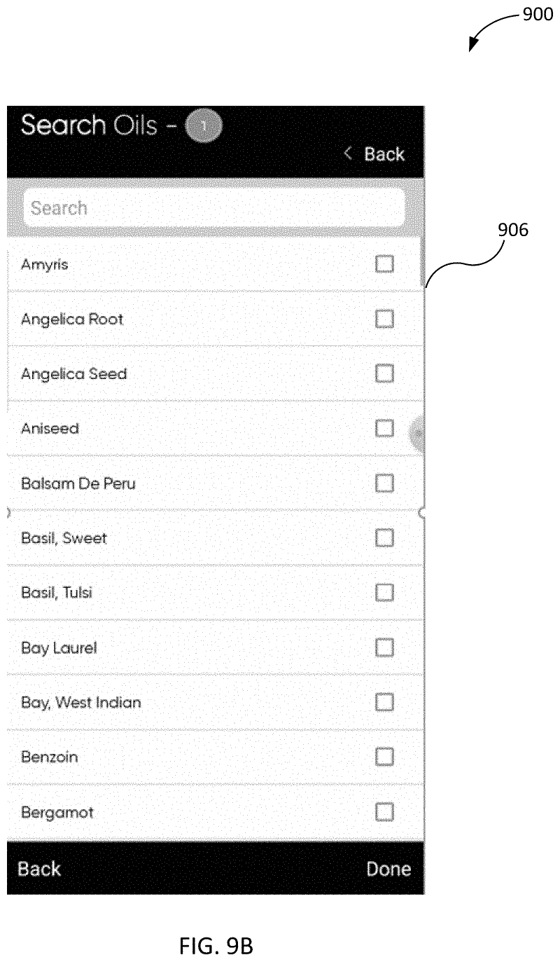

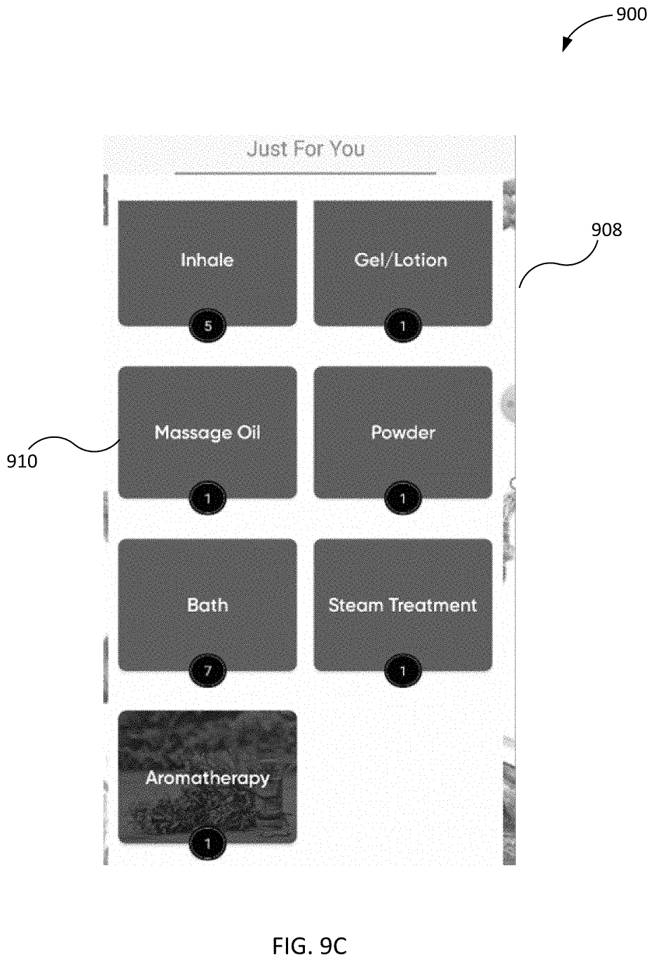

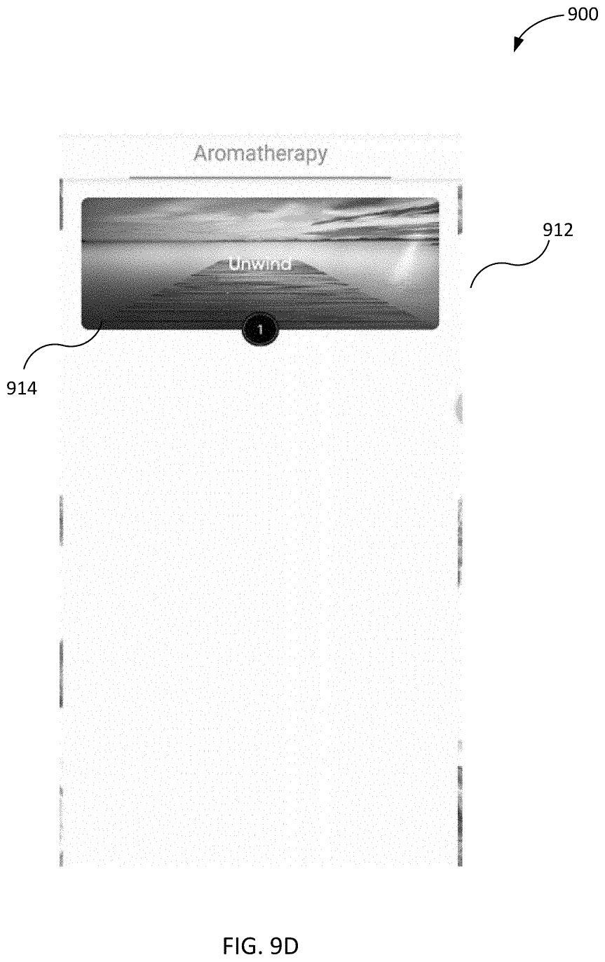

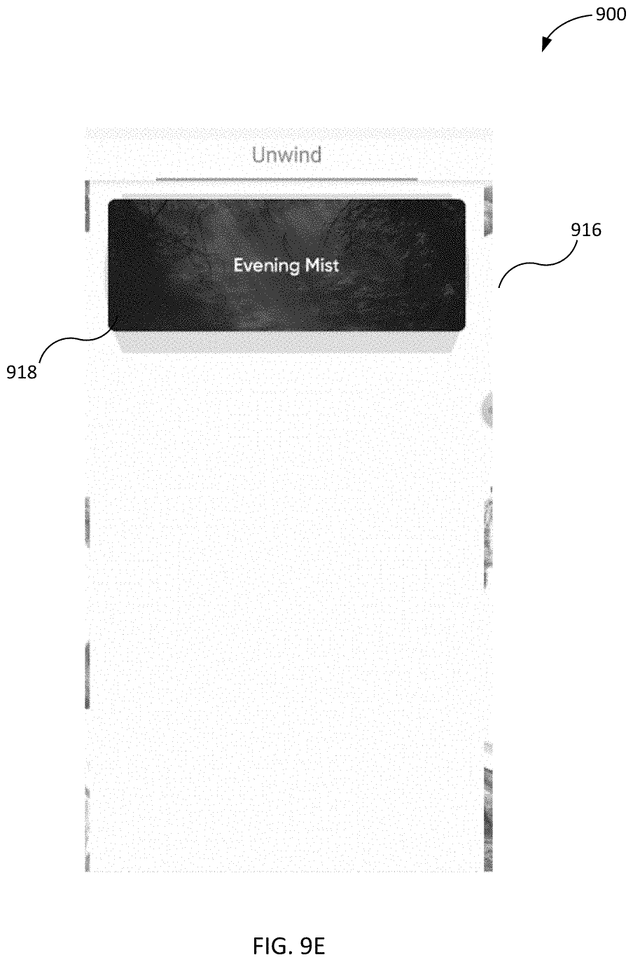

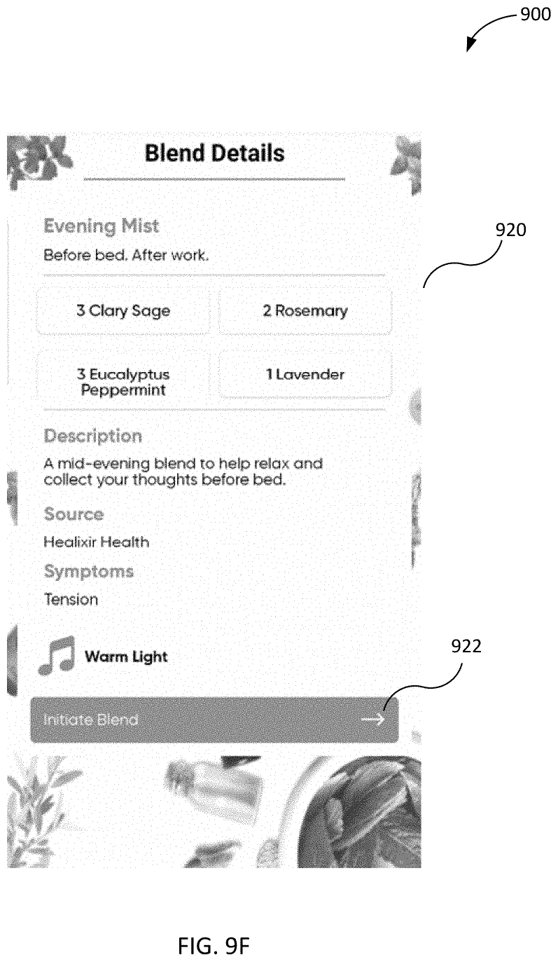

As noted above, the diffuser 100 may dispense one or more oils based on a recipe that specifies which oils and in what quantities to dispense to create a particular scent and/or other fluid characteristic. The recipes may be provided to the controller in several ways. For example, in some embodiments, a user may input the recipes into the controller using the user interface of the diffuser 100. In some embodiments, the user may input the recipes using a remote device, such as via a mobile application. Use of a remote device to input oil information and/or scent recipes to the controller is described in greater detail in relation to FIGS. 9A-9F. In other embodiments, the recipes may be stored on a remote server and accessed using a menu provided on the interface of the diffuser 100 and/or via the user's mobile device.

In some embodiments, the diffuser 100 may be programmed to mix a particular scent at a particular time of day. For example, the diffuser 100 may be used as part of a wake up plan to produce an early morning scent at a particular time prior to, at, and/or after a user plans to wake up. For example, a scent recipe may be mixed and diffused prior to, during, and/or after an alarm clock and/or other device being used to wake the user is activated, allowing the diffused scent to help gradually wake the user. As another example, a calming scent recipe may be mixed and diffused just prior to, during, and/or after a user's typical bedtime to help the user drift asleep. Other types of scents and/or timing for automatically operating the diffuser 100 may be contemplated in accordance with the present invention.

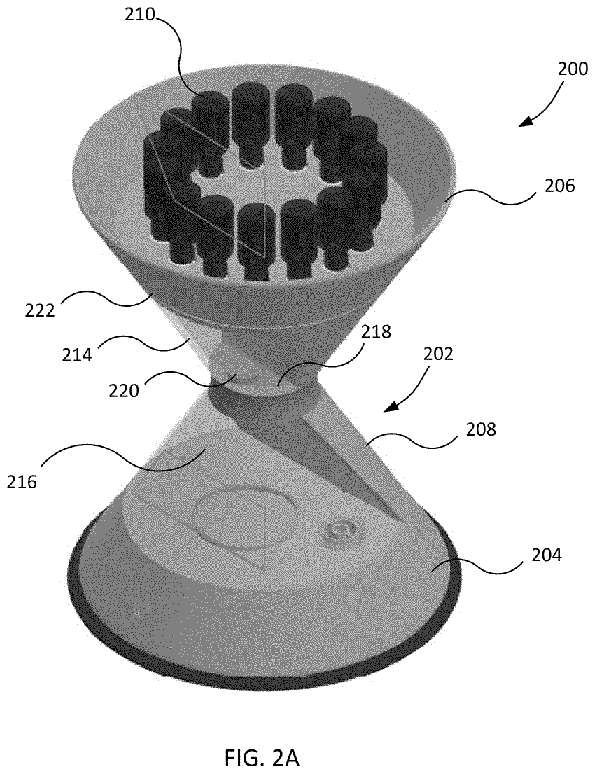

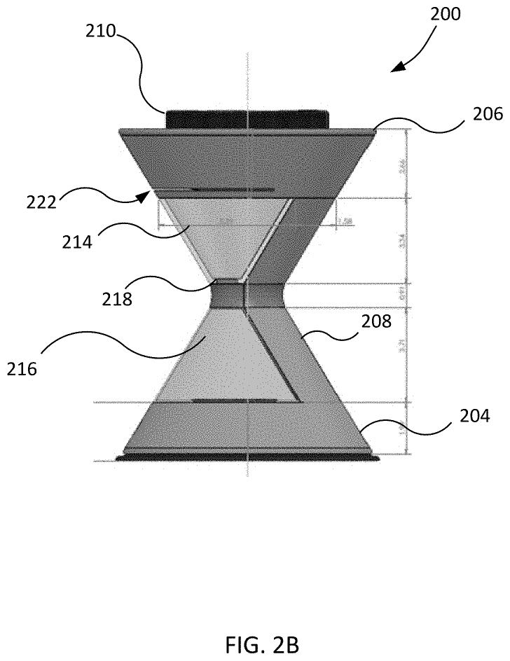

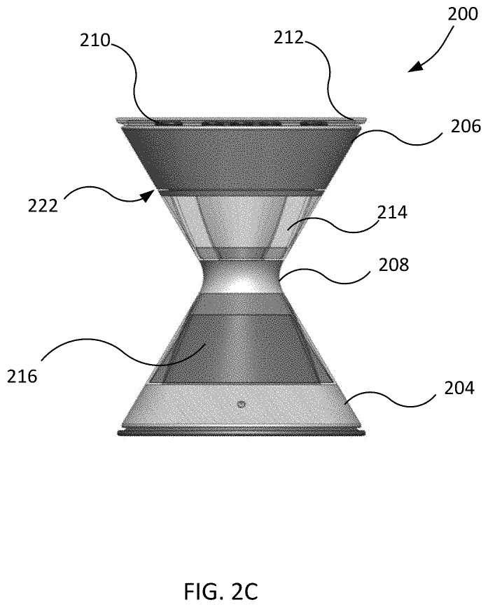

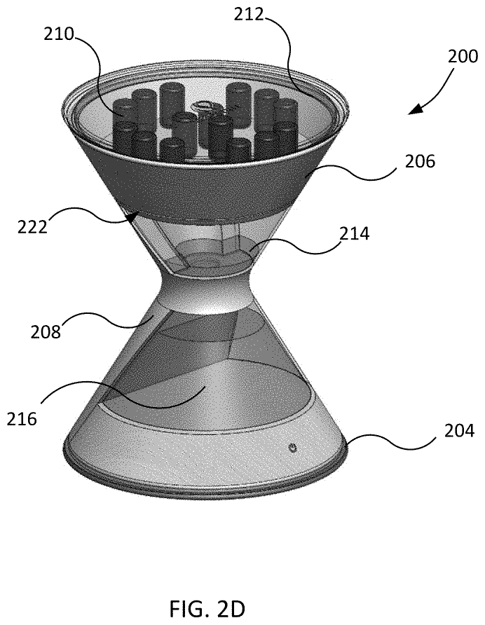

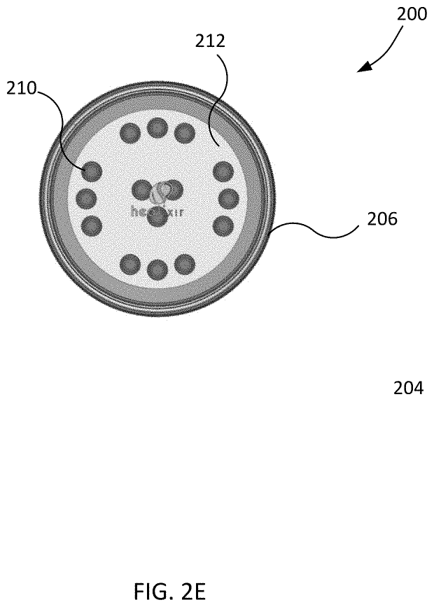

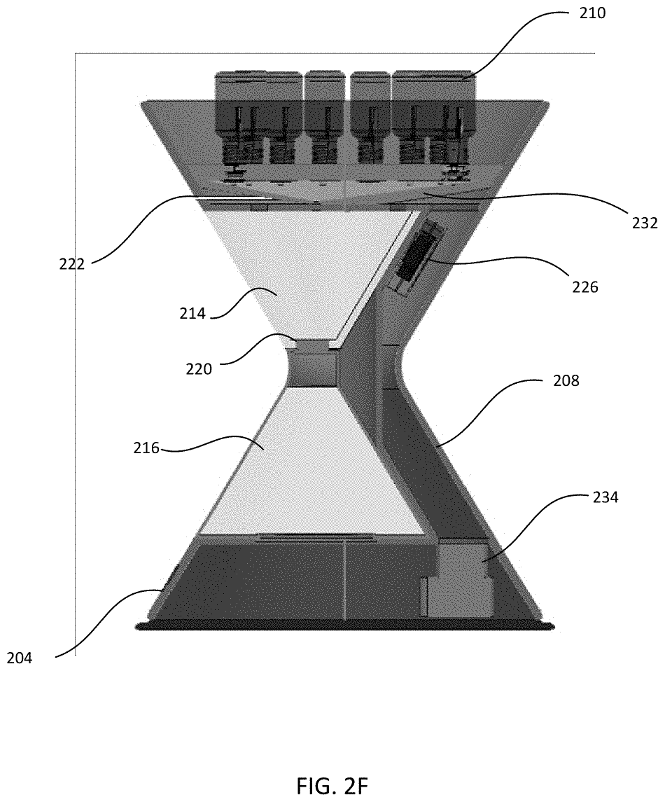

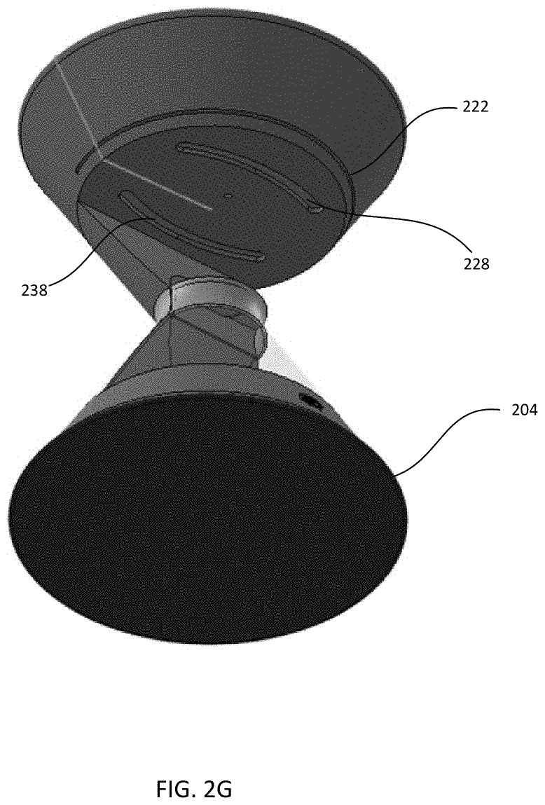

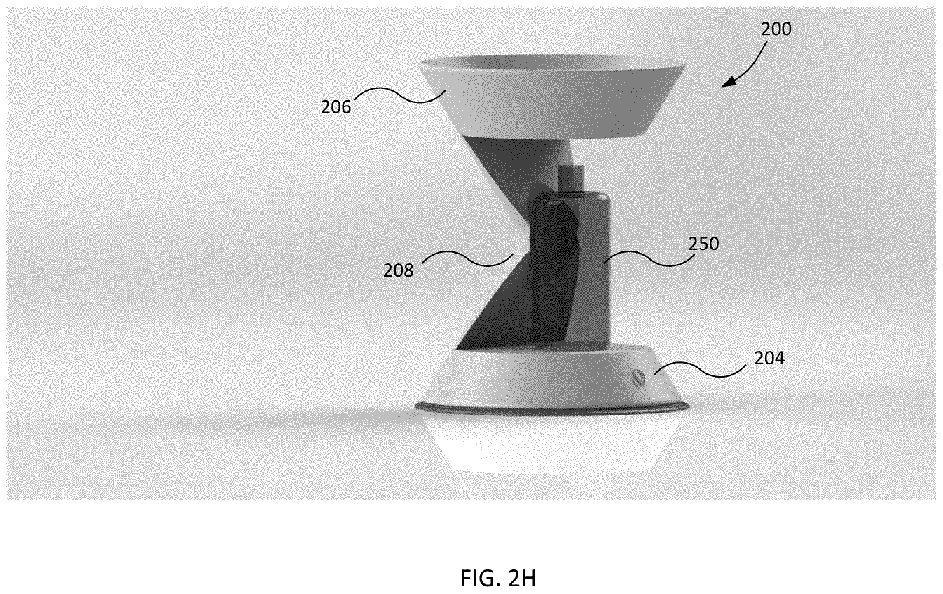

FIGS. 2A-2G depict another embodiment of an oil diffuser 200. Oil diffuser 200 may be similar to oil diffuser 100 above, and may include any of the features thereof. The diffuser 200 may include a housing 202. As illustrated, the housing 202 includes a base 204 that is connected to a dispensing portion 206 using a spine 208 that extends vertically between the base 204 and the dispensing portion 206. The dispensing portion 206 may define a number of fluid ports (shown with a fluid dispenser 210 interfaced within each fluid port) that are configured to receive and/or otherwise interface with a fluid dispenser 210. In some embodiments, the fluid ports may be similar to those described in relation to oil diffuser 100. Each fluid dispenser 210 may include a container and a dispensing valve, such as the valves described in relation to FIGS. 4A-7D. In some embodiments, the fluid port may include a connector that is used to secure a fluid dispenser within the fluid port. For example, a snap connector, threaded connector, magnet, and/or other coupling mechanism may be provided within the fluid port. In other embodiments, the fluid dispensers may be held within the fluid ports using only gravity. It will be appreciated that any number of fluid ports may be positioned on the oil diffuser 200 in any arrangement, with greater numbers of fluid ports being able to produce a greater variety of oil blends. In some embodiments, a lid or other cover 212 may be positioned atop the dispensing portion 206 when access to the fluid ports and/or fluid dispensers 210 is not necessary as best illustrated in FIG. 2C.

In some embodiments, each of the fluid ports may include an electrical connector (not shown). Each electrical connector may be configured to interface with a corresponding connector of a valve of a fluid dispenser received therein. In some embodiments, the electrical connector may be positioned on a side of the fluid port and/or formed in a side of the fluid port. In other embodiments, the electrical connector may be at or near a base of the fluid port. Once interfaced with a corresponding connector of a fluid dispenser 210, the electrical connector may enable the oil diffuser 200 to provide power and/or instructions to the fluid dispenser 210 that controls the operation of a valve within the fluid dispenser 210. As just one example, a controller (not shown) of the oil diffuser 200 may send a signal that causes a valve of the fluid dispenser 210 to open and/or close to dispense a metered volume of an essential oil stored within the fluid dispenser 210.

Each fluid port may define a fluid path that delivers oils to a mixing basin 214. The mixing basin 214 may be positioned below the fluid ports such that when oils are dispensed from the fluid dispensers 210, the oils flow into the mixing basin 214. As best illustrated in FIG. 2E, in some embodiments diffuser 200 may include a funnel 232 that helps direct the dispensed oils into the mixing basin 214. In some embodiments, the funnel 232 may be coated with a substance that helps prevent the oil from sticking on the surface of the funnel 232, such as an oleo-phobic coating. The mixing basin 214 may hold a volume of water (or other fluid) in which the oils are dispensed to form a mixture. In some embodiments, the mixing basin 214 may be coupled with one or more fluid sources. For example, in some embodiments the oil diffuser 200 may include a water reservoir 216 that is in fluid communication with the mixing basin 214 such that a volume of water may be transferred from the water reservoir 216 to the mixing basin 214. In some embodiments, the volume of water may be supplied via one or more pumps 234 and/or valves disposed within the housing 202. For example, pump 234 may draw water from the water reservoir 216 via a port 236 provided in the base 204. The water may be pumped up the spine 208 where it is deposited into the mixing basin 214 via a port 238 (best illustrated in FIG. 2F). In some embodiments, the water reservoir 216 may be removably coupled with the housing 202 such that the water reservoir 216 may be removed for easier filling and/or cleaning. In some embodiments, rather than, or in addition to, having a water reservoir 216, the oil diffuser 200 may include an additional water port that may be coupled directly with an incoming water line that may supply the mixing basin 214 and/or water reservoir 216 with a volume of water when a valve is actuated.

The mixing basin 214 may include a base plate 218 that supports the mixture of water and oils. In some embodiments, the walls of the mixing basin 214 may taper inward such that the mixture is directed downward toward an atomizer 220 that is positioned at or near a lowest portion of the base plate 218. The atomizer 220 may then receive at least a portion of the mixture of fluid and atomize the mixture into an atomized vapor or mist. The atomizer 220 may be an ultrasonic atomizer, a heat atomizer, evaporative atomizer, and/or a nebulizer.

As best illustrated in FIG. 2E, the oil diffuser 200 may also include at least one fan 226 and/or other air flow device. The fan 226 is configured to push and/or otherwise draw the air containing the atomized mixture out of the air diffuser 200 via a ventilation port 228 formed in the housing 202 near a top of the mixing basin 214. The atomized particles of the mixture of oils and water may be entrained within the moving air generated by the fan and transported out of the diffuser 200.

In some embodiments, a removable vial or other container may be interfaced with the diffuser 200 to collect mixtures of various oils dispensed via the fluid ports. For example, in some embodiments the mixing basin 214 may be removed from the housing 202 such that the vial may be positioned atop the base plate 218. This allows volumes of mixed recipes of oils to be dispensed, collected, and retained for topical treatments and/or later use.

The diffuser 200 may also include a controller (not shown) that is configured to control the operation of the fluid ports (and fluid dispensers 210 interfaced therewith), the atomizer 220, and the fan. For example, circuitry may be used to connect the controller with the various components. Additionally, as disclosed above, the controller may be coupled with the fluid dispensers 210 via the electrical connectors, which allows the controller to actuate the valves of the various fluid dispensers 210. For example, actuation of valves of the various fluid dispensers 210 may then dispense a volume of oil through one of the fluid ports. The controller may activate a particular set of valves based on a particular scent recipe. As disclosed above, once the oils are dispensed according to the appropriate recipe, they are deposited in the mixing basin 214 and/or the removable mixing tray for diffusion and/or other use. If the oils are dispensed into the mixing basin 214, the controller may then send commands to the atomizer 220 and/or fan to diffuse the oils and expel the oils and water mixture into the air outside of the diffuser 200. The controller may operate similar to the controller described in relation to diffuser 100.

In some embodiments, both the mixing basin and water reservoir 216 may be removably coupled with the housing 202. In such embodiments, the diffuser 200 may be used to dispense a blend of one or more oils (or other liquids) into a base product. For example, a bottle 250 (or other container) of a shampoo base, conditioner base, lotion base, etc., may be positioned beneath the fluid ports and atop the base 204 of the housing 202 as illustrated in FIG. 2G. The user may then select a blend of oils to be dispensed into the bottle. This allows the blend of oil to contribute to the properties (taste, smell, color, healing properties, etc.) of the base substance. The user may then create customized products that include properties of the various oil blends they prefer.

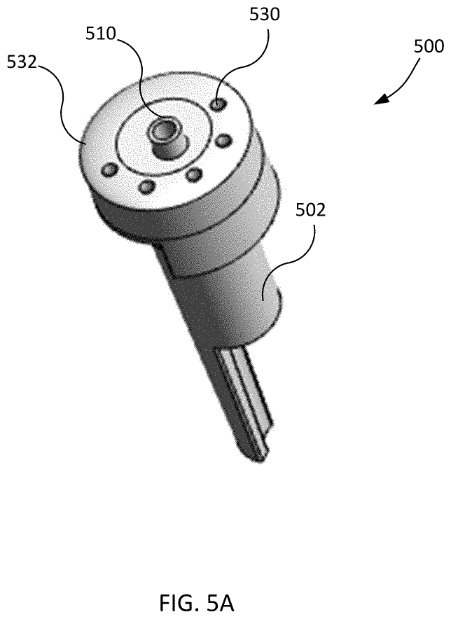

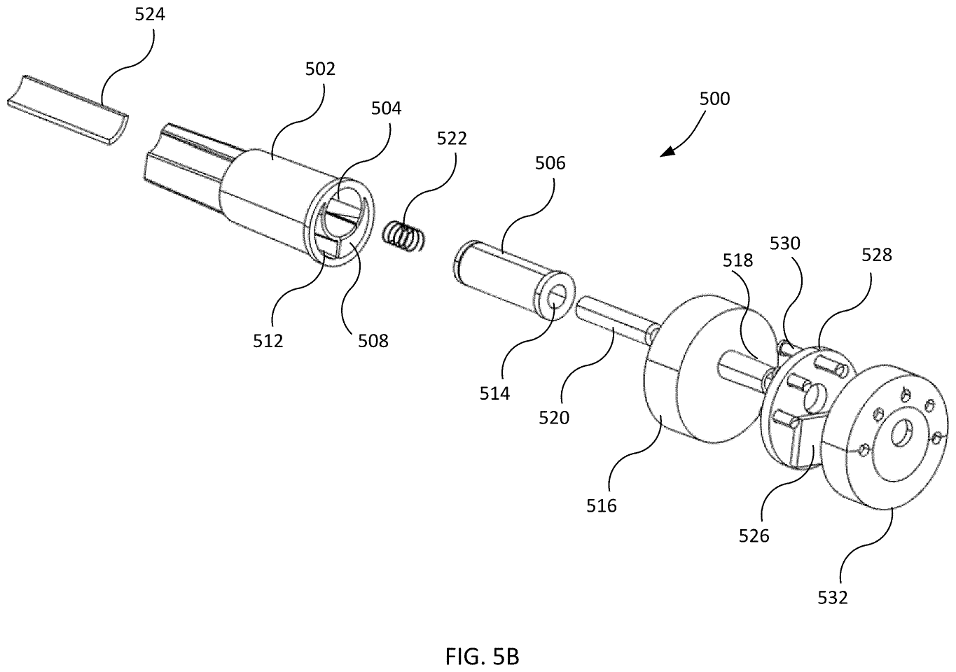

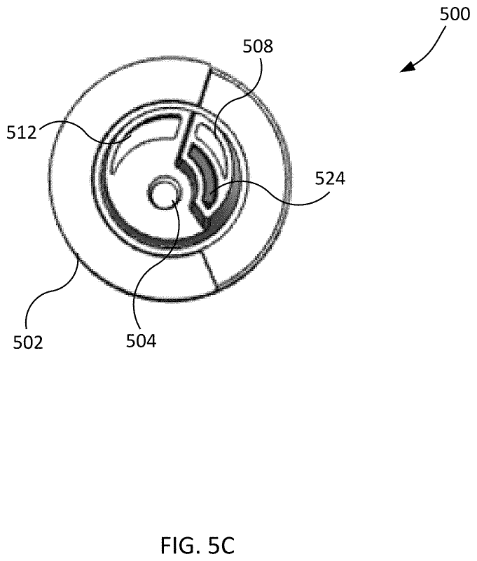

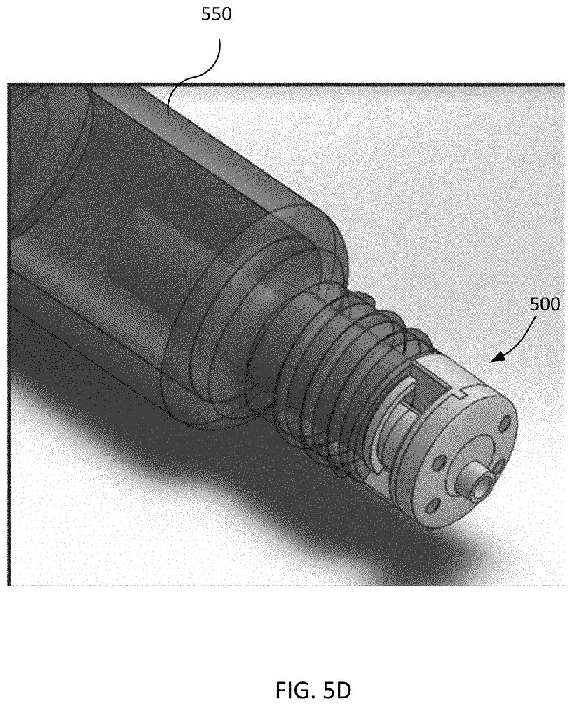

FIGS. 3A-3F depict another embodiment of an oil diffuser 300. Oil diffuser 300 may be similar to oil diffuser 100 and/or 200 described above and may include any of the features thereof. For example, the diffuser 300 may include a housing 302. As illustrated, the housing 302 includes a base 304 that is connected to a dispensing portion 306. The dispensing portion 306 may define a number of fluid ports 308 that are each configured to receive and/or otherwise interface with a fluid dispenser 310. In some embodiments, the fluid ports 308 may be similar to those described in relation to oil diffuser 100. Each fluid dispenser 310 may include a container and a dispensing valve, such as the valves described in relation to FIGS. 5A-7D. In some embodiments, the fluid port 308 may include a connector that is used to secure a fluid dispenser 310 within the fluid port 308. For example, a snap connector, threaded connector, magnet, and/or other coupling mechanism may be provided within the fluid port 308. In other embodiments, the fluid dispensers 310 may be held within the fluid ports 308 using only gravity. It will be appreciated that any number of fluid ports 308 may be positioned on the oil diffuser 300 in any arrangement, with greater numbers of fluid ports 308 being able to produce a greater variety of oil blends.

In some embodiments, each of the fluid ports 308 may include an electrical connector (not shown). Each electrical connector may be configured to interface with a corresponding connector of a valve of a fluid dispenser 310 received therein. In some embodiments, the electrical connector may be positioned on a side of the fluid port 308 and/or formed in a side of the fluid port 308. In other embodiments, the electrical connector may be at or near a base of the fluid port 308. For example, each electrical connector may include a number of pogo pins that are aligned and interfaced with a corresponding electrical connectors of a dispenser 310 when the fluid dispenser 310 is positioned within the fluid port 308. Once interfaced with a corresponding connector of a fluid dispenser 310, the electrical connector may enable the oil diffuser 300 to provide power and/or instructions to the fluid dispenser 310 that controls the operation of a valve within the fluid dispenser 310. As just one example, a controller (not shown) of the oil diffuser 300 may send a signal that causes a valve of the fluid dispenser 310 to open and/or close to dispense a metered volume of an essential oil (or other fluid) stored within the fluid dispenser 310. The electrical connectors may include connectors for communicating with an ID reader (such as an RF reader), a water level sensor, a wifi-enabled, atomizer connection, microcontroller, capacitive sensors, indicator lights, and/or other electronic devices.

Each fluid port 308 may define a fluid path that delivers oils to a mixing basin 314 (as best shown in FIGS. 3C-3E). The mixing basin 314 may be positioned below the fluid ports 308 such that when oils are dispensed from the fluid dispensers 310, the oils flow downward into the mixing basin 314. The mixing basin 314 may receive and/or hold a volume of water (or other fluid) in which the oils are dispensed to form a mixture. In some embodiments, the mixing basin 314 may be coupled with one or more fluid sources. For example, in some embodiments the oil diffuser 300 may include a water reservoir 316 that is in fluid communication with the mixing basin 314 such that a volume of water may be transferred from the water reservoir 316 to the mixing basin 314. In some embodiments, the volume of water may be supplied via one or more pumps 334, fluid conduits 350, and/or valves disposed within the housing 202. For example, water from the water reservoir 316 may drain downwards to a pump 334, which may pump the water from the water reservoir 316 through conduit 350, and into the mixing basin 314. In some embodiments, the water reservoir 316 may be removably coupled with the housing 302 such that the water reservoir 316 may be removed for easier filling and/or cleaning. In some embodiments, rather than, or in addition to, having a water reservoir 316, the oil diffuser 300 may include an additional water port that may be coupled directly with an incoming water line that may supply the mixing basin 314 and/or water reservoir 316 with a volume of water when a valve is actuated.

At least a portion of the mixture of water and oils may be drained, pumped, and/or otherwise delivered from the mixing basin 314 to an atomizer 320, which may atomize the mixture into an atomized vapor or mist. The atomizer 320 may be an ultrasonic atomizer, a heat atomizer, evaporative atomizer, and/or a nebulizer. The oil diffuser 300 may also include at least one fan and/or other air flow device that may push and/or otherwise draw the air containing the atomized mixture out of the air diffuser 300 via a ventilation port 328 that is in fluid communication with the atomizer 320. The atomized particles of the mixture of oils and water may be entrained within the moving air generated by the fan and transported out of the diffuser 300.

The diffuser 300 may also include a controller (not shown) that is configured to control the operation of the fluid ports (and fluid dispensers 310 interfaced therewith), the atomizer 320, and/or the fan. For example, circuitry may be used to connect the controller with the various components. Additionally, as disclosed above, the controller may be coupled with the fluid dispensers 310 via the electrical connectors, which allows the controller to actuate the valves of the various fluid dispensers 310. For example, actuation of valves of the various fluid dispensers 310 may then dispense a volume of oil through one of the fluid ports. The controller may activate a particular set of valves based on a particular scent recipe. As disclosed above, once the oils are dispensed according to the appropriate recipe, they are deposited in the mixing basin 314 and/or the removable mixing tray for diffusion and/or other use. If the oils are dispensed into the mixing basin 314, the controller may then send commands to the atomizer 320 and/or fan to diffuse the oils and expel the oils and water mixture into the air outside of the diffuser 300. The controller may operate similar to the controller described in relation to diffusers 100 and 200.

In some embodiments, the diffuser 300 may be self-cleaning to clean the atomizer 320 and/or other components, such as by decalcifying and/or otherwise demineralizing the diffuser 300. For exapmle, a fluid dispenser 310 having a cleaning solution, which may be a specially formulated cleaning agent and/or a solution such as white vinegar and water, may be provided within one of the fluid ports 308. The controller of the diffuser 300 may programmed to conduct a self-cleaning process on demand and/or based on a periodic schedule (once a day, week, month, etc.). In operation, the self-cleaning process may involve dispensing a volume of the cleaning solution from the fluid dispenser 310 and delivering the solution to the mixing basin 314 and atomizer 320. The atomizer 320 may atomize the cleaning solution to remove any minerals that may have accumulated on the atomizer 320.

FIGS. 4A-4C illustrate one embodiment of a valve 400, which may be used in conjunction with diffuser 100, 200, or 300, such as by being inserted within one of the fluid ports. Valve 400 may include a valve body 402 that is configured to be inserted at least partially into an interior of a container (not shown) that stores a fluid, such as an oil. For example, in some embodiments, the valve body 402 may be press fit, friction fit, and/or otherwise received within an opening and/or neck of the container, and may operate to dispense a metered volume of fluid from the container each time the valve 400 is actuated. In some embodiments, the valve body 402 may be a single piece, while in other embodiments the valve body may be formed from multiple components that are joined together. The valve body 402 may define a fluid lumen 404 that extends along at least a portion of the length of the valve body 402. The fluid lumen 404 may provide a path for fluid from the container to pass through the valve 400 and out via a valve tip 406 that is in fluid communication with the fluid lumen 404.

The valve 400 may also include an actuator 408 that is used to control operation of the valve 400. In some embodiments, the actuator 408 may be positioned within the valve body 402, such as within fluid lumen 404. As illustrated here, the actuator 408 is a solenoid, however other types of actuators may be utilized in some embodiments. In some embodiments, the solenoid may define a central opening that allows fluid to flow through the solenoid and the fluid lumen 404. A plunger 412 may be positioned within the valve body 402 and configured to seal and/or otherwise close the valve tip 406. For example, the plunger 412 may be positioned within the fluid lumen 404 and/or within a central opening of the actuator 408, with an end of the plunger 412 being positioned against an opening of the valve tip 406 to close off the valve tip 406 and prevent any fluid from escaping the valve 400. The actuator 408 may be coupled with and configured to move the plunger 412, such as by reciprocating the plunger 412, to open the valve tip 406 to allow a metered volume of fluid to be dispensed from the valve 400. In embodiments in which the actuator 408 is a solenoid, the solenoid may receive an electric current that generates a magnetic field that pushes, pulls, and/or otherwise moves the plunger 412 (which may include a ferromagnetic material) away from the valve tip 406 to allow the fluid to be dispensed. In some embodiments, to maintain the plunger 412 in a sealing position against the valve tip 406 when not opened by the actuator 408 a biasing element, such as a spring (not shown) may be positioned such that the spring pushes, pulls, and/or otherwise biases the plunger 412 against the valve tip 406.

The valve 400 may include a fluid level sensor 414. The fluid level sensor 414 may be configured to determine a volume of fluid that is contained within the container with which the valve 400 is interfaced and/or whether a sufficient amount of fluid is present for a particular scent recipe. The fluid level sensor 414 may be disposed within the valve body 402 and/or within the fluid lumen 404 such that the fluid level sensor 414 is in contact with any fluid within a neck of the container in which the valve 400 is interfaced. In some embodiments, the fluid level sensor 414 may be a capacitive sensor that measures changes in capacitance to determine a fluid level and/or volume of the fluid within the container. In such embodiments, a length of the capacitive sensor may determine when the sensor begins detecting variations in the volume of fluid within the container, as the sensor will begin detecting volume changes when an oil level of the container falls below a distalmost tip of the capacitive sensor. For example, a capacitive sensor that extends to or nearly to a base of the container will begin detecting volume changes nearly immediately upon oil being dispensed from the container, while in embodiments with a short capacitive sensor, the sensor will sense a full container until the oil level falls below the tip of the sensor.

The valve 400 may also include a valve cap 416 that defines an opening that terminates in the valve tip 406. As illustrated, the valve cap 416 coupled with the valve body 402 and helps retain the actuator 408 and plunger 412 within the valve body 402. In some embodiments, the valve cap 416 may include a number of electronic leads 418, which may be at least partially exposed through a surface of the valve cap 416. This allows the electronic leads 418 to be connected to the electrical connectors of the diffuser 100 and/or other device. The electronic leads 418 enable power and/or commands to be received by the valve 400 to control operation of the actuator 408 and/or the fluid level sensor 414. While shown here with four electronic leads 418 positioned on a front face of the valve cap 416, it will be appreciated that any number of electronic leads 418 may be positioned anywhere on the valve cap 416 and/or the valve body 402. In some embodiments, to help orient the valve 400 properly within a device, such as diffuser 100, the valve cap 416 and/or valve body 402 may include a number of magnetic elements 420 that attract metallic features in the device, which may help maintain contact between the electrical leads 418 and the electrical connectors.

Another embodiment of a valve 500 is depicted in FIGS. 5A-5E. Valve 500 may include similar features and specifications as valve 400 and may include any of the features thereof, and may be used in conjunction with diffuser 100, 200, or 300, such as by being inserted within one of the fluid ports. For example, valve 500 may include a valve body 502 that is configured to be inserted at least partially into an interior of a container (not shown) that stores a fluid, such as an oil. In some embodiments, the valve body 502 may be a single piece, while in other embodiments the valve body may be formed from multiple components that are joined together. As illustrated, the valve body 502 may define three lumens. For example, a first lumen 504 is configured to receive an actuator 506. A second lumen 508 is a fluid lumen that extends along at least a portion of the length of the valve body 502 and provides a path for fluid to flow from a distal end of the valve body 502 to a proximal end of the valve body 502 and out via a valve tip 510 that is in fluid communication with the fluid lumen 504. A third lumen 512 provides an air return channel to relieve the vacuum created due to the fluid flowing out of the valve body 502. In some embodiments, some or all of the lumens may be offset from a central axis of the valve body 502.

The second lumen 508 may deliver the dispensed fluid to an oil cap 516, which defines a channel 518 that provides a fluid path that terminates in the valve tip 510. The oil cap 516 seals off the second lumen 508 from electronic components of the valve 500, thereby ensuring that any dispensed fluid passing through the valve 500 does not contact the electronic components. In some embodiments, the channel 518 defines an opening positioned opposite the valve tip 510. The opening may be positioned in alignment with the first lumen 504 and a surface of the oil cap 516 may be sloped toward the opening such that fluid dispensed from the second lumen 508 is directed into the opening and through the valve tip 510.

As indicated above, the valve 500 may include an actuator 506 that is used to control operation of the valve 500. As illustrated here, the actuator 506 is a solenoid, however other types of actuators may be utilized in some embodiments. In some embodiments, the solenoid may define a central opening 514 that receives a plunger 520. An end of the plunger 520 may be positioned against the opening of the oil cap 516 such that the plunger 520 may close the opening to prevent fluid from exiting the valve tip 510 until the actuator 506 is operated. The actuator 506 may be coupled with and configured to move the plunger 520, such as by reciprocating the plunger 520, to provide access to the opening of the oil cap 516 and the valve tip 510 to allow a metered volume of fluid to be dispensed from the valve 500. In embodiments in which the actuator 506 is a solenoid, the solenoid may receive an electric current that generates a magnetic field that pushes, pulls, and/or otherwise moves the plunger 520 (which may include a ferromagnetic material) away from the opening of the oil cap 516 to allow the fluid to be dispensed. In some embodiments, to maintain the plunger 520 in a sealing position against the opening of the oil cap 516 when not opened by the actuator 506 a biasing element, such as a spring 522 may be positioned such that the spring pushes, pulls, and/or otherwise biases the plunger 520 against the opening of the oil cap 516. For example, as illustrated, spring 522 is positioned within the first lumen 504 and/or within the actuator 506 and presses against an end of the plunger 520 opposite the opening of the oil cap 516 to keep the channel 518 sealed. When the actuator 506 is activated, the plunger 520 is pulled away from the opening of the oil cap 516 with a force that exceeds the force of spring 522 to open the valve 500 to dispense a volume of fluid from the container.

The valve 500 may include one or more fluid level sensors 524. The fluid level sensor 524 may be configured to determine a volume of fluid that is contained within the container with which the valve 500 is interfaced and/or whether a sufficient amount of fluid is present for a particular scent recipe. The fluid level sensor 524 may be coupled with the valve body 502 such that a portion of the fluid level sensor 524 is in contact with any fluid within a neck and/or body of the container in which the valve 500 is interfaced. For example, as best illustrated in FIG. 5D, the fluid level sensor 524 is positioned within a portion of the valve body 502 that projects into the body of the container 550 such that an exposed distal end of the fluid level sensor 524 is in contact with fluid provided within the container 550. It will be appreciated that the fluid level sensor 524 may be positioned differently in some embodiments. In some embodiments, the fluid level sensor 524 may be a capacitive sensor that measures changes in capacitance to determine a fluid level and/or volume of the fluid within the container. For example, the capacitive sensor may be in the form of two copper plates that are positioned side by side that may be used to test the capacitance of the contents (oil or other fluid) of the container. The capacitance of the contents changes based on the volume of the contents, allowing the fluid level and/or volume to be determined.

The valve 500 may include a controller 526 that includes one or more processors. The controller 526 may be configured to perform several functions. For example, the controller 526 may trigger the operation (opening and/or closing) of the actuator 506 to dispense a metered volume of fluid from the valve 500, determine the volume and/or level of the fluid within the container based on data from the fluid level sensor 524, and/or may store information associated with the fluid (type, viscosity, etc.) and/or its usage. This information may be passed to an external device, such as diffuser 100 or 200. In some embodiments, the controller 526 may be mounted on a printed circuit board (PCB) 528. The PCB 528 may include other circuitry necessary to operate the valve 500. Additionally, the PCB 528 may include a number of electronic leads 530, which allow the valve 500 to be electrically coupled with an external device, such as the diffuser 100 using the electrical connectors. The electronic leads 530 enable power and/or commands to be received by the valve 500 to control operation of the actuator 506 and/or the fluid level sensor 524. While shown here with five electronic leads 530 positioned on a front face of the PCB 528, it will be appreciated that any number of electronic leads 530 may be positioned anywhere on the valve 500. In some embodiments, the electronic leads 530 may be in the form of pogo pins that extend from a face of the PCB 528. This allows the valve 500 to be dropped and/or otherwise placed within the fluid ports of the diffuser without damaging the electronic leads 530 and/or other portion of the valve 500.

The valve 500 may also include a valve cap 532 that defines an opening that is configured to receive the channel 518 and valve tip 510. As illustrated, the valve cap 532 coupled with the valve body 502 and helps retain the actuator 506, plunger 520, oil cap 516, and PCB 528 within the valve body 502. In some embodiments, the valve cap 532 may define a number of openings 534 that are configured to receive the electronic leads 530, which once inserted within the openings 534 may be at least partially exposed through a surface of the valve cap 532.

As indicated above, the valve 500 may be removably coupled with a bottle or other container. This allows the valve 500 to be used with any number of containers and/or allows the containers to be refilled for reuse. Oftentimes, the valve body 502 is constructed to be sized and shaped to fit existing oil bottles with a friction fit. This allows the valve 500 to be quickly and easily inserted into and removed from a conventional bottle, enabling users to utilize any existing oil product with the diffusers described herein. FIGS. 5D and 5E illustrate the valve 500 being interfaced with a container 550.

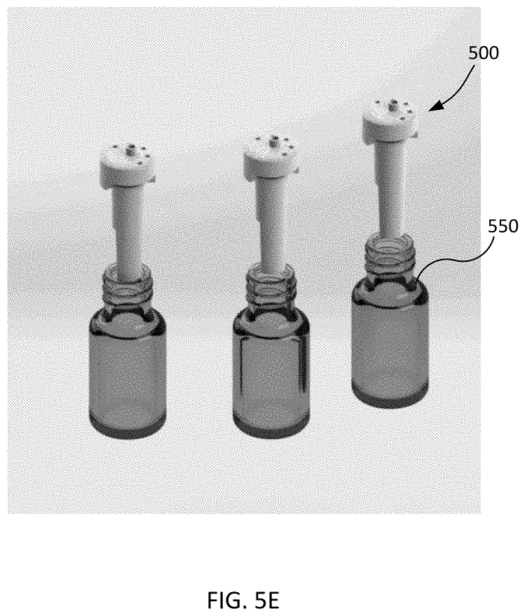

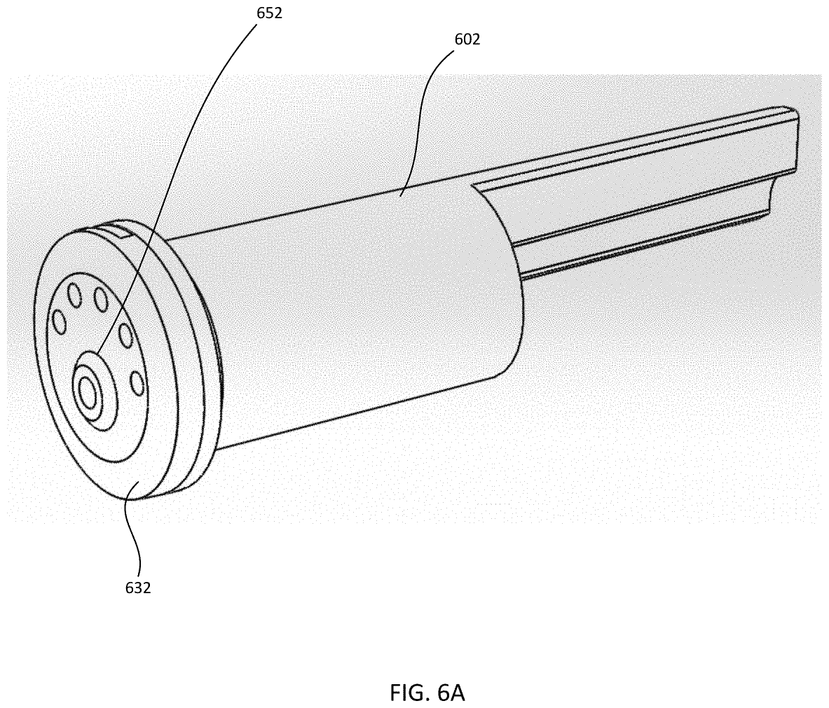

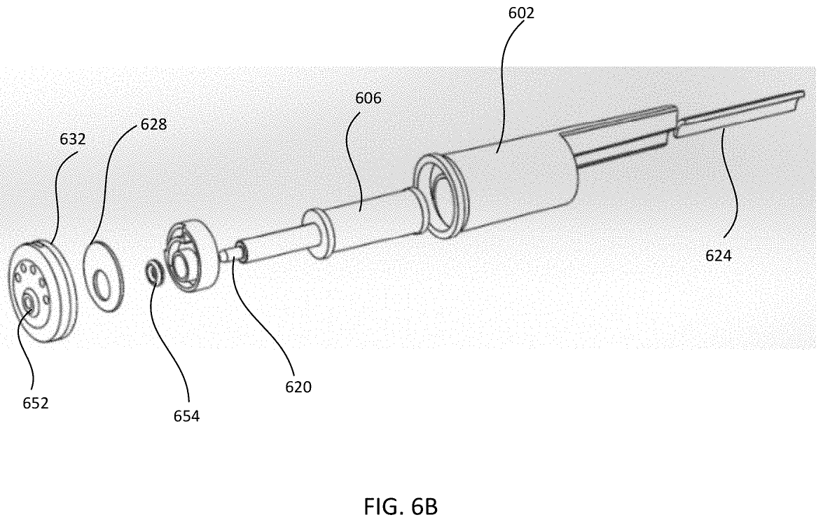

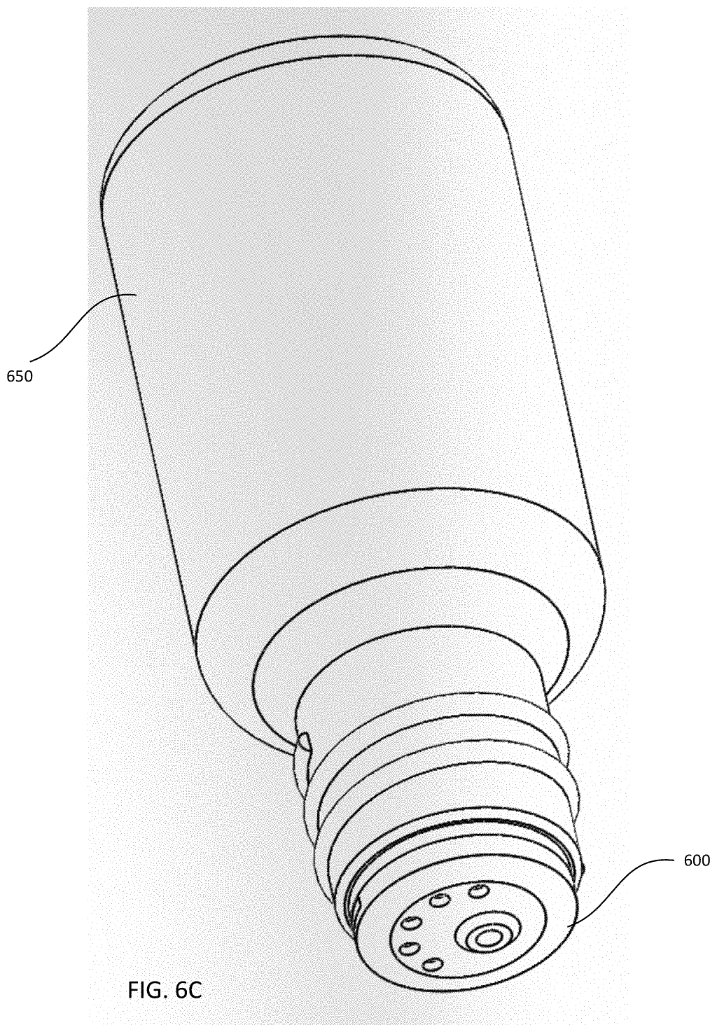

Another embodiment of a valve 600 is depicted in FIGS. 6A-6C. Valve 600 may include similar features and specifications as valves 400 and 500 and may include any of the features thereof, and may be used in conjunction with diffuser 100, 200, or 300, such as by being inserted within one of the fluid ports. For example, valve 600 may include a valve body 602 that is configured to be inserted at least partially into an interior of a container 650 (shown in FIG. 6C) that stores a fluid, such as an oil. In some embodiments, the valve body 602 may be a single piece, while in other embodiments the valve body may be formed from multiple components that are joined together. Similar to valve 400, the valve body 602 may define three lumens: a first to receive an actuator 606, a second acts as a fluid lumen that extends along at least a portion of the length of the valve body 602 and provides a path for fluid to flow from a distal end of the valve body 602 to a proximal end of the valve body 602 and out via a valve opening 610 that is in fluid communication with the fluid lumen, and a third that provides an air return channel to relieve the vacuum created due to the fluid flowing out of the valve body 602. In some embodiments, some or all of the lumens may be offset from a central axis of the valve body 602.

The fluid lumen may deliver the dispensed fluid to an oil cap 616, which defines a channel 618 that provides a fluid path that terminates in the valve tip opening 610. The oil cap 616 seals off the fluid lumen from electronic components of the valve 600, thereby ensuring that any dispensed fluid passing through the valve 400 does not contact the electronic components. The valve opening 610 may be positioned in alignment with the first lumen and a surface of the oil cap 616 may be sloped toward the valve opening 610 such that fluid dispensed from the fluid lumen is directed into the valve opening 610 and through a valve tip 652 that is formed in a valve cap 632.

As indicated above, the valve 600 may include an actuator 606 that is used to control operation of the valve 600. As illustrated here, the actuator 606 is a solenoid, however other types of actuators may be utilized in some embodiments. In some embodiments, the solenoid may define a central opening that receives a plunger 620. An end of the plunger 620 may extend through the valve opening 610 and may be positioned against an interior surface of the valve tip 652 of the valve cap 632 such that the plunger 620 may close the opening to prevent fluid from exiting the valve tip 652 until the actuator 606 is operated. In some embodiments, an O-ring 654 and/or other sealing mechanism is provided to further seal the interface between the valve tip 652 and plunger 620. The actuator 606 may be coupled with and configured to move the plunger 620, such as by reciprocating or otherwise translating the plunger 620, to provide access to the valve tip 652 to allow a metered volume of fluid to be dispensed from the valve 600. In some embodiments, to maintain the plunger 620 in a sealing position against the valve tip 652 when not opened by the actuator 606 a biasing element, such as a spring (not shown) may be positioned such that the spring pushes, pulls, and/or otherwise biases the plunger 620 against the valve tip 652.

The valve 600 may include one or more fluid level sensors 624. The fluid level sensor 624 may be configured to determine a volume of fluid that is contained within the container with which the valve 600 is interfaced and/or whether a sufficient amount of fluid is present for a particular scent recipe. The fluid level sensor 624 may be coupled with the valve body 602 such that a portion of the fluid level sensor 624 is in contact with any fluid within a neck and/or body of the container in which the valve 600 is interfaced. For example, as best illustrated in FIG. 6C, the fluid level sensor 624 is positioned within a portion of the valve body 602 that projects into the body of the container 650 such that an exposed distal end of the fluid level sensor 624 is in contact with fluid provided within the container 650. It will be appreciated that the fluid level sensor 624 may be positioned differently in some embodiments. In some embodiments, the fluid level sensor 624 may be a capacitive sensor that measures changes in capacitance to determine a fluid level and/or volume of the fluid within the container. For example, the capacitive sensor may be in the form of two copper plates that are positioned side by side that may be used to test the capacitance of the contents (oil or other fluid) of the container. The capacitance of the contents changes based on the volume of the contents, allowing the fluid level and/or volume to be determined.

The valve 600 may include a controller (not shown) that includes one or more processors. The controller may be configured to perform several functions. For example, the controller may trigger the operation (opening and/or closing) of the actuator 606 to dispense a metered volume of fluid from the valve 600, determine the volume and/or level of the fluid within the container based on data from the fluid level sensor 624, and/or may store information associated with the fluid (type, viscosity, etc.) and/or its usage. This information may be passed to an external device, such as diffuser 100 or 200. In some embodiments, the controller may be mounted on a printed circuit board (PCB) 628. The PCB 628 may include other circuitry necessary to operate the valve 600. In some embodiments, rather than having a controller on the valve 600 itself, the controller of the dispensing device/diffuser may control the operation of the valve 600 via the PCB 628. For example, the dispensing device/diffuser may include its own PCB and controller which connect to the valve 600 via electronic leads (such as pogo pins or other connectors) that are provided on the valve 600. In some embodiments, the device PCB may supply one or more voltages to the valve 600 via one or more of the electronic leads. For example, voltages of 24V and 3V3 may be supplied to the valve. By supplying multiple voltages to the valve 600, the need for a voltage regulator on the valve PCB 628 may be eliminated. The electronic leads may also include connections for the valve ground (such as for the actuator 606), a fluid sensor ground, controlling and/or communicating information about the valve level, and/or other features. In some embodiments, the actuator 606 may receive power through the 24V lead. Such a configuration may enable a field-effect transistor (FET) (which may control the flow of current to the valve 600) to the main device PCB instead of the valve PCB 628, which may improve heat dissipation, power delivery, and reduce the cost of the valve PCB 628. In some embodiments, the fluid sensor lead may be an analog output that may use a 555 timer configured as a capacitive level sensor. The 555 timer may powered by the 3V3 lead and may use a separate ground lead. Using such a PCB topology, the need for a valve microcontroller may be eliminated, with all valve actuation being managed by the main microcontroller of the diffuser/dispenser which may greatly simplify the valves and the overall topology of the device while boosting performance and cutting cost.

The valve 600 may also include a valve cap 632 that seals the valve 600 and terminates in the valve tip 652 through which the fluid is dispensed. As illustrated, the valve cap 632 coupled with the valve body 602 and helps retain the actuator 606, plunger 620, oil cap 616, O-ring 654 and PCB 628 within the valve body 602. In some embodiments, the valve cap 632 may define a number of openings 634 that are configured to receive electronic leads, which once inserted within the openings 634 may be at least partially exposed through a surface of the valve cap 632.

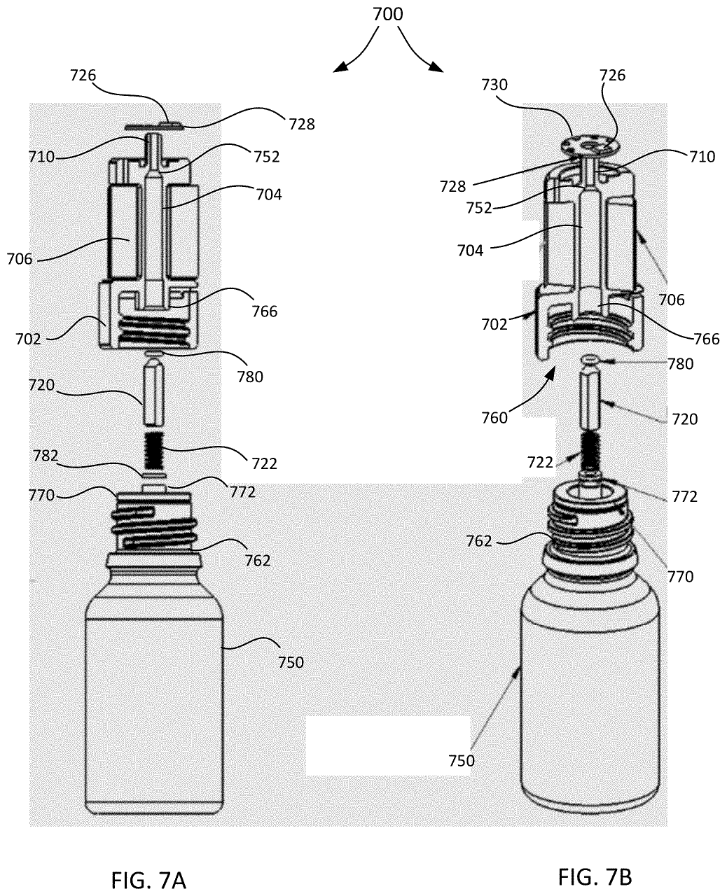

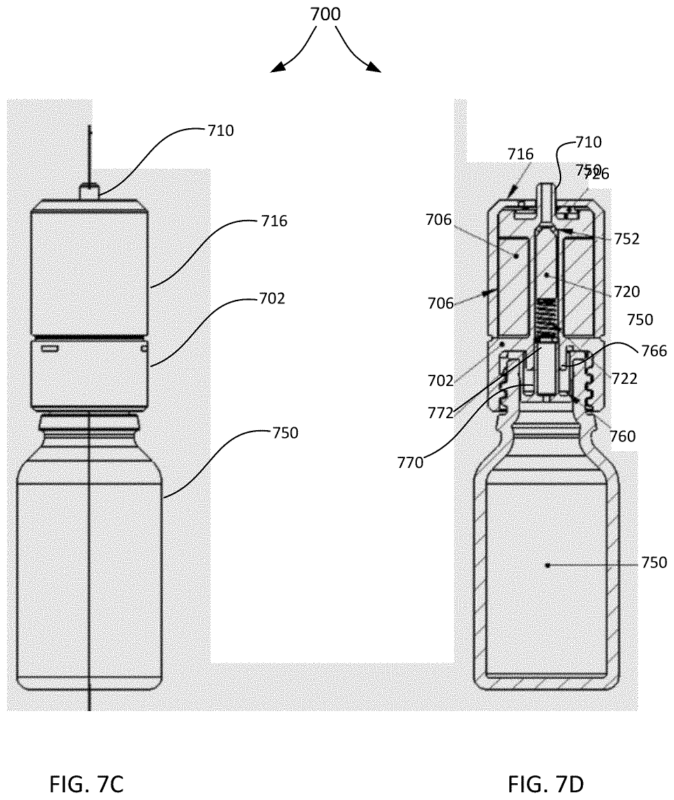

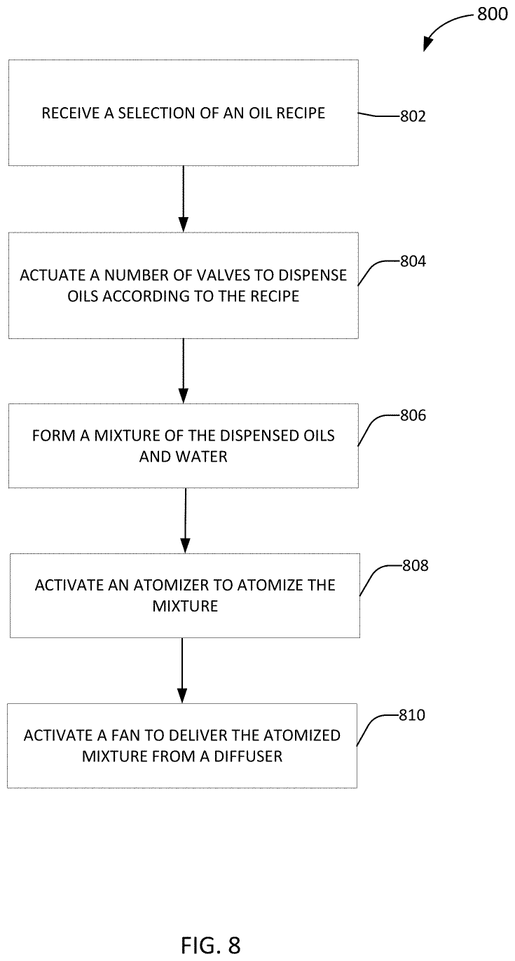

FIGS. 7A-7D illustrate another embodiment of a valve 700. Valve 700 may include similar features and specifications as valves 400, 500, and 600 and may include any of the features thereof, and may be used in conjunction with diffuser 100, 200, or 300, such as by being inserted within one of the fluid ports. For example, valve 700 may include a valve body 702 that is configured to interface with a container 750 that stores a fluid, such as an oil or other liquid. For example, the valve body 702 may define a recess 760 that is configured to receive and secure a neck 762 or other outlet of the container 750 with the valve body 702. In some embodiments, a fastening mechanism, such as internal threading, may be used to secure the valve body 702 onto the container 750. Such a threaded (or other fastener) connection may enable the valve 700 to be interfaced with any existing dispensing container (including those which include Euro droppers and/or other existing dispensing devices), enabling any number of existing fluid containers to be used with the diffusers and/or other devices described herein. Additionally, such a design enables the valves 700 to be reusable with other containers 750 once a given container 750 is empty.

In some embodiments, the valve body 702 may be a single piece, while in other embodiments the valve body may be formed from multiple components that are joined together. As illustrated, the valve body 702 may define a fluid lumen 704 that extends along at least a portion of the length of the valve body 702 and provides a path for fluid to flow from a distal end of the valve body 702 to a proximal end of the valve body 702 and out via a valve tip 710 that is in fluid communication with the fluid lumen 704. In some embodiments, air may enter the fluid lumen 704 via the valve tip 710 which causes a non-laminar more drop like effect within the valve 700. In other embodiments, the valve body 702 may define a separate air return channel to relieve the vacuum created due to the fluid flowing out of the valve body 702. In some embodiments, some or all of the lumens may be offset from and/or coaxial with a central axis of the valve body 702.

As indicated above, the valve 700 may include an actuator 706 that is used to control operation of the valve 700. As illustrated here, the actuator 706 is a solenoid, however other types of actuators may be utilized in some embodiments. In some embodiments, the solenoid may define a central opening that receives a plunger 720. An end of the plunger 720 may be positioned against a plunger seat 752 disposed near the valve tip 710 such that the plunger 720 may close an opening of the plunger seat 752 to prevent fluid from exiting the valve tip 710 until the actuator 706 is operated. In some embodiments, the plunger 720 and/or the plunger seat 752 may include an O-ring 780 and/or other sealing member that may help better seal the opening of the plunger seat 752 when the plunger 720 is in the closed position. The actuator 706 may be coupled with and configured to move the plunger 720, such as by reciprocating the plunger 720, to provide access to the valve tip 710 via the opening of the plunger seat 752 to allow a metered volume of fluid to be dispensed from the valve 700. For example, in some embodiments, the fluid may pass through the fluid lumen 704, flow around the plunger 720 and through the opening of the plunger seat 752 when the plunger 720 is moved to an open position. In other embodiments, the fluid may pass through a separate lumen that delivers the fluid to the plunger seat 752 such as described above in relation to valves 500 and 600.

In embodiments in which the actuator 706 is a solenoid, the solenoid may receive an electric current that generates a magnetic field that pushes, pulls, and/or otherwise moves the plunger 720 (which may include a ferromagnetic material) away from the plunger seat 752 to allow the fluid to be dispensed. In some embodiments, to maintain the plunger 720 in a sealing position against the plunger seat 752 when not opened by the actuator 706 a biasing element, such as a spring 722 may be positioned such that the spring pushes, pulls, and/or otherwise biases the plunger 720 against the plunger seat 752. For example, as illustrated, spring 722 is positioned within the fluid lumen 704 and/or within the actuator 706 and presses against an end of the plunger 720 opposite the plunger seat 752 to keep the opening of the plunger seat 752 sealed. When the actuator 706 is activated, the plunger 720 is pulled away from the opening of the plunger seat 752 with a force that exceeds the force of spring 722 to open the valve 700 to dispense a volume of fluid from the container. In some embodiments, a washer 782 and/or other insert may be fixed in a position behind the spring 722 to provide a backing that may enable the spring 722 to compress when the plunger 720 is moved into the open position. The spring's compressive force may then bias the plunger 720 against the plunger seat 752 when the actuator 706 is not being actuated to seal the opening of the plunger seat 752.

The valve 700 may include a controller 726 that includes one or more processors. The controller 726 may be configured to perform several functions. For example, the controller 726 may trigger the operation (opening and/or closing) of the actuator 706 to dispense a metered volume of fluid from the valve 700, determine the volume and/or level of the fluid within the container based on data from the fluid level sensor, and/or may store information associated with the fluid (type, viscosity, etc.) and/or its usage. This information may be passed to an external device, such as diffuser 100, 200, or 300 and/or other dispenser and/or mixing device. In some embodiments, the controller 726 may be mounted on a printed circuit board (PCB) 728. The PCB 728 may include other circuitry necessary to operate the valve 700. Additionally, the PCB 728 may include a number of electronic leads 730, which allow the valve 700 to be electrically coupled with an external device, such as a diffuser, using the electrical connectors. The electronic leads 730 enable power and/or commands to be received by the valve 700 to control operation of the actuator 706 and/or any sensors. For example, the electronic leads 730 may include a number of leads for interfacing with a controller of the diffuser (or other device) and/or a number of leads for coupling the actuator and/or any sensors. In some embodiments, the electronic leads 730 may be in the form of connection ports or pads that interface with corresponding pins or other connectors on the diffuser or other external device. In some embodiments, the electronic leads 730 may be in the form of pogo pins that extend from a face of the PCB 728 that interface with ports or pads on an external device.

An outer housing 716 may fit around the valve body 702 and internal components and may seal off the fluid path from electronic components of the valve 700 (such as PCB 728, controller 726, and electronic leads 730), thereby ensuring that any dispensed fluid passing through the valve 700 does not contact the electronic components. The valve tip 710 may extend through the outer housing 716 to enable fluid to be ejected from the valve 700. The outer housing 716 may snap and/or otherwise fasten onto the valve body 702. In some embodiments, the outer housing 716 may define a number of openings (not shown) that are configured to provide access to the electronic leads 730.

In some embodiments, the valve 700 may include one or more fluid level sensors. In some embodiments, the sensors may include capacitive sensors as described above. In some embodiments, the sensors may include flow sensors that can determine how much fluid has been dispensed from the container 750 via the valve 700. In such embodiments, a fluid level of the container 750 may be determined based on the dispensed volume of fluid and the initial fluid level in the container 750. This determination may be made using the controller 726 and/or using a controller on an external device, such as a diffuser, which may have information about each container 750 and/or fluid as described elsewhere herein. In such embodiments, the valve 700 and/or diffuser (or other device) may include a sensor or other device that monitors how much fluid has been dispensed from a given container 750. This information may be reset each time the container 750 is replaced with a new container 750, which may or may not be the same size, shape, and/or contain the same fluid. In other embodiments, fluid level determinations may be performed by a diffuser or other dispenser, such as by using load sensors and/or other sensors to determine a volume of fluid remaining in the container 750 based on a known initial weight of the filled container 750 and a known weight per volume/drop of the fluid.

In some embodiments, a Euro dropper 770 (or other dispensing device) may be disposed between the valve 700 and the outlet of the container 750. For example, the Euro dropper 770 may be provided within the neck of the container 750, with the neck and the Euro dropper 770 being received within the recess 760 of the valve body 702. In some embodiments, a tip 772 of the Euro dropper 770 may be received within an interior receptacle 766 of the valve body 702, while in other embodiments, the tip 772 may be freely disposed within an interior of the recess 760 and/or other portion of the valve body 702. In some embodiments, the interior receptacle 766 may be sized and shaped to seal the connection between the outlet of the Euro dropper 770 and the interior receptacle 766. In some embodiments, an additional sealing member, such as an O-ring or gasket may be included to further seal this interface.

While several embodiments of valves for fluid dispensers are described above, it will be appreciated that other types of valves and/or fluid dispensers may be used in accordance with the present invention. For example, a Euro dropper may be used to dispense fluids from the fluid dispenser. In some embodiments, the valves may be permanently affixed to a container, while in other embodiments, the valves may be removable. This allows the containers to be refilled. In such embodiments, the container may be filled with the same kind of oil, or a different type of oil may be used. In some embodiments, if a container is filled with a different kind of oil, the controller of the valve may be programmed with oil information (such as a type of oil, viscosity, etc.) associated with the new oil. In some embodiments, this may be done by interfacing the valve with a device such as diffuser 100, 200, or 300 that has a communications interface that allows a user to input information either via the diffuser directly (such as using a button and/or other interface of the diffuser and/or via a mobile application on a remote device that is interfaced with the diffuser).

In some embodiments, the operation of the actuator of each valve may be based on the type of oil or other fluid being dispensed. For example, each oil may have its own fluid properties, such as viscosity. As such, an opening distance and/or duration of valve actuation may be modified based on the characteristics of the oil in order to dispense a proper volume of the fluid. As such, when a scent recipe calls for a particular amount of a particular oil, the controller of the diffuser and/or the controller of the respective valve may determine an opening distance and/or duration of the valve based on a volume of fluid to be dispensed, a viscosity of the fluid, and/or other physical characteristics of the fluid. For example, each valve and/or diffuser may be programmed with an algorithm which takes in the viscosity and the amount of oil to dispense as parameters and outputs an amount of time to open the valve to dispense the desired quantity of oil. The eeprom on the valve may contain the viscosity numbers in order to successfully run the algorithm. For example, in the case of Eucalyptus, which is a more viscous oil, the valve may be open for a slightly longer duration than a less viscous oil like Lavender, to achieve the same amount of metered fluid.

In order for the valves described herein to be useable with existing oil containers (which often have very narrow necks) while still being able to repeatedly dispense metered volumes of oils and/or other viscous fluids, the valves must be designed with certain operating characteristics. For example, in embodiments using solenoids, the solenoids may have between approximately 125 and 350 turns of wire, with between 150 and 250 more common, and often about 185 turns. Typically, the solenoid is charged with between about 0 and 20 volts, with between 5-12 volts being most common. At this voltage, the solenoid can generate up to about 20 mN of force, with approximately 25 mN/volt for a plunger that is formed of 3CrCh, although different materials of plungers may result in different forces, up to about 40 mN/volt. The solenoid is typically between about 5 and 25 mm in length, with lengths of 10-20 mm more common, and often about 14.5 mm. An inner diameter of the solenoid is typically between about 2-7 mm, with an inner diameter of about 4 mm being most common. An outer diameter of the solenoid may be between about 3 and 11 mm, often between about 5 and 8 mm, with about 6 mm being most common. In some embodiments, the solenoid may be steel-wrapped, with a thickness of the steel being between about 0.01 and 0.1 inches. Oftentimes, the plunger may be between about 6 mm and 25 mm in length, more often between about 10 mm and 15 mm. In some embodiments, the plunger may have a constant diameter, while in other embodiments, the plunger may have a tapered tip that engages with the opening of the valve cap and/or plunger seat. In embodiments using an O-ring, the O-ring may have a durometer of between about 10-60 Shore A, and may often formed from silicone. In some embodiments, the O-ring may have an inner diameter of between about 1 and 2 mm, with 1.5 mm being most common. The O-ring may have a thickness of between about 0.5 and 1.5 mm, with 1 mm being most common. The O-ring may have an outer diameter of between about 2 mm and 5 mm, with about 3.5 mm being most common. Oftentimes, the capacitive sensor may be formed from at least two coplanar plates. Each plate may be between about 3 and 8 mm in width, with 5 mm being most common. Each plate may have a length of between about 10 and 25 mm, with about 18 mm being most common, however it will be appreciated that longer capacitive sensors may be used to provide more data as the deeper into the container body the capacitive sensor reaches, the earlier the sensor may provide indications of changes in the volume of the container. The capacitive sensor will often be configured to sense down to the pico-farad. It will be appreciated that the specifications described above are described in relation to valves that are being utilized in standard 2-15 mL essential oil containers that have neck diameters of about 10 mm. In other embodiments, dispenser/diffuser devices may utilize valves having different sized components.

For example, for larger bottles (such as 100, 150, 250, 500, 1000 mL, etc.) the dimensions (in particular the outer diameter of the valve) may be modified to fit the respective bottle or container.

FIG. 8 is a flowchart illustrating a process 800 of operating an oil diffuser. Process 800 may utilize a diffuser similar to diffuser 100, 200, 300 and/or dispensing containers that utilize valves similar to valves 400, 500, 600, and 700 described above. Process 800 may begin at block 802 by receiving a selection of an oil recipe. The oil recipe may include instructions for a volume of each of a plurality of essential oils needed to make a particular scent and/or other fluid characteristic. The oil recipe may be selected using a user interface of a diffuser and/or from a remote device, such as via mobile application that is in communication with a communications interface of the diffuser. In some embodiments, the recipe may be stored on a memory device of the diffuser and/or the remote device such that the user only needs to make a selection of an existing scent recipe. In some embodiments, the user may select a scent recipe from a server using the mobile application and/or the diffuser. In other embodiments, the user may manually input a scent recipe using a user interface of the diffuser and/or the remote device.

Once the scent recipe has been selected, a valve of each of a number of oil dispensers may be actuated to dispense the volume of each of the plurality of essential oils at block 804. The actuation of each valve may be determined based at least in part on the oil recipe. For example, if a particular oil recipe calls for particular quantities (which may be the same or different) of three different oils, the valves of the dispensers associated with each of these three oils may be actuated to dispense the desired volume of each of the three oils. In some embodiments, the actuation of each valve is based at least in part on a viscosity of the essential oil being dispensed, as the viscosity effects how quickly the oil flows and what size of opening is required for the oil to pass through. As a result, the controller of the diffuser and/or valve may adjust a duration of actuating the valve and/or a size of the opening of the valve based on the viscosity of the type of oil being dispensed and/or volume to be dispensed.

After the various oils are dispensed, a mixture of the dispensed essential oils and water may be formed at block 806. For example, the dispensed oils may be dispensed into a mixing basin of the diffuser where the oils may mix with water. In some embodiments, the water may be supplied from a water source, such as a water tank of the diffuser and/or a water supply line. Once the oil and water are mixed, an atomizer may be activated at block 808 to atomize at least a portion of the mixture. For example, an atomizer may be present within the mixing basin that operates to atomize some or all of the mixture within the mixing basin. A fan or other airflow device may then be activated that moves air through the mixing basin to deliver the atomized mixture out of a ventilation port of the oil diffuser at block 810. In some embodiments, the fan may be positioned within the mixing basin, while in other embodiments the fan may be in a separate area of the diffuser, such as a ventilation chamber.

In some embodiments, rather than mixing the dispensed oils with water and atomizing the mixture, the oils may be dispensed in a removable tray of the diffuser. This allows the dispensed oils to mix to form a scent recipe that may be saved for a future use in a diffuser, topical usage, and/or other purpose. For example, the tray may be removed and the mixed oils may be poured into a container, such as a bottle for future use and/or application. In other embodiments, the oils may be dispensed directly into another container, such as a container containing shampoo, lotion, coffee, soda, and/or other substance. In such a manner, the oil mixtures to add scents, flavors, and/or other ingredients to another substance.

FIGS. 9A-9F illustrate a user interface of a mobile application 900 that allows a user to control a diffuser and/or valves (such as diffuser 100, 200, 300 and/or valves 400, 500, 600, 700) using a remote device. As illustrated in FIG. 9A, the mobile application 900 may include a screen 902 that allows a user to view and/or assign a particular type of oil to a particular slot of a diffuser device. For example, to assign an oil to a slot, a user may interact with an icon 904 for a particular slot. In some embodiments, this may cause the mobile application 900 to navigate to screen 906 as shown in FIG. 9B. Screen 906 may include a directory of known oils that may be searched and/or browsed to locate the oil that is present within a particular slot of the diffuser. Once the user locates the correct oil, the user may select the oil to assign it to a particular slot. In some embodiments, if the oil present in the slot is not found in the directory, the user may type in a custom oil name.

Once the mobile application 900 has been initialized with the oils that are present within the diffuser, the user may access a screen 908 that provides a menu of icons 910 for oil categories that are available to mix. For example, as illustrated in FIG. 9C the oil categories may include, without limitation, inhalation recipes, gel/lotion recipes, massage oil recipes, powder recipes, bath recipes, steam treatment recipes, aromatherapy recipes, and/or other types of oil recipes. Once a user selects a category, a screen 912 that includes one or more subcategories and/or recipes may be displayed. In the embodiment illustrated in FIG. 9D, screen 912 includes subcategories. For example, under the aromatherapy category, a subcategory for scent recipes for unwinding are displayed. The user may select an icon 914 associated with the desired subcategory, which may cause screen 916 to be displayed. As illustrated in FIG. 9E, screen 916 includes a number of scent recipes that fall within the particular subcategory. A user may select a desired scent recipe icon 918. The user may then be presented with screen 920, which may include information associated with the selected recipe. As shown in FIG. 9F, screen 920 may include information about how the blend is typically used (e.g., before bed or after work, etc.), the recipe itself (often in terms of parts, drops, or volumes of each oil and/or volume of water), a description of the blend itself, a source of the recipe, symptoms the recipe may treat, a recommended music and/or light conditions, and/or other information. It will be appreciated that other information may be presented on screen 920. Screen 920 may also include an icon 922 that when selected, causes the diffuser to mix and/or diffuse the selected recipe.

In some embodiments, the mobile application 900 may also allow the user to schedule a recipe to be mixed and/or diffused at a particular time and/or date. The mobile application 900 may allow the user to control lights, sounds (music, white noise, etc.), alarm clocks and/or other features that may be integrated into and/or otherwise connected with the diffuser. It will be appreciated that the various screens of mobile application 900 are provided merely as one example of a mobile application that may be used in conjunction with the diffusers described herein and that variations will exist. For example, more or less functionality may be built into the mobile applications. Additionally, in some embodiments the functionality of some or all of the screens may be integrated into a single screen and/or broken up into multiple screens. Additionally, while described as a mobile application, it will be appreciated that in some embodiments, the mobile application 900 may be provided as software for a user interface that is integrated into a body of the diffuser itself.

While described largely in the context of dispensing essential oils, coloring agents, and/or other scents, it will be appreciated that the invention is not so limited. The valves and/or techniques described herein may be utilized in numerous other applications. In such embodiments, rather than being placed into a diffuser, the dispensing valves described herein may be used in conjunction with dispensing mechanisms and/or mixing devices that do not diffuse any oils but that operates in a similar manner to the diffusers described herein to dispense blends or recipes of oils and other fluids. For example, the dispensing devices may include fluid ports that may receive any number of dispensing mechanisms/valves. Controllers on the device and/or valves may enable a user to dispense and/or mix precise quantities of one or more fluid ingredients, which may be collected by hand and/or in a dispensing tray, bottle, and/or other container. In some embodiments, the device may include an interface (attached or wireless, such as using a separate computing device) that allows users to select a recipe, quantity, and/or other feature of the oil to be dispensed. The user may place a container under the fluid dispensers and have oils and/or other fluids mixed and subsequently collected by the container. In some embodiments, the container may include a base solution, such as a lotion or shampoo base, and the dispensed fluids may be additives, including colors, scents, and the like. In some embodiments, the device may also include one or more payment readers, such as RF readers, magnetic stripe readers, optical readers such as barcode readers, and the like. The user may then interact with the payment reader and/or user interface to pay for any oil blends dispensed. It will be appreciated that except where explicitly described in relation to diffusion actions (atomizing, venting, etc.), features described in relation to diffusers may be incorporated into other dispensing/mixing devices and that the terms dispensing and/or mixing devices and diffusers may be used interchangeably. In some instances, such dispensing/mixing devices may be used in commercial applications.

In one particular embodiment, the valves and/or techniques may be utilized to dispense liquid multi-vitamins and/or supplements. Such applications may solve significant problems in the health and wellness fields, as 40% of Americans have extreme difficulty or an inability to swallow pills. Additionally, liquid vitamins and/or supplements have a 90% greater absorption rate than pills, and typically have no sugar, fillers, or preservatives, unlike tablets/pills. In some embodiments, custom nutrition plans and/or wellness plans may be managed through the dispensing device and/or mobile application by recommending a personalized benefit profile of particular liquid supplements/vitamins that are specially dosed based on various metrics and health goals desired by the user. These metrics may include demographic information about the user and/or health data about the user. Adhering to a routine can be simplified by automating a schedule for the intake of daily vitamins/supplements throughout the day. Additionally, the problem of seniors remembering to take daily vitamins/supplements can be resolved through daily reminders in the app. By utilizing smart valves (such as those described above) to dispense these medications and/or supplements in liquid form, dispensed volumes can be metered with precision to ensure safe and effective doses that splitting pills does not provide. Just as done for essential oils, the valves may be inserted into containers of any number of liquid vitamins (such as, but not limited to, turmeric, taurine, green tea extract, B-vitamins, cinnamon, etc.). In some embodiments, multi-vitamin blends (which may include combinations such as, but not limited to, energy “shots” or sleep combinations) may be accessed via an app, concocted by a mixing device by triggering the dispensing of metered doses of one or more medications, vitamins, supplements, and/or other substances (possibly including flavoring or sweetener substances), funneled through into a coffee cup, protein shake bottle, glass vial, or any other preferred medium. The app may ensure safety by metering the level of each ingredient based on suggested use by the manufacturer. This information may be stored in the e-prom chip, RFID tag on the ingredient container, and/or a remote server and may be regulated by the software. In some embodiments, back-end data analytics may be used to recommend new ingredients and/or track use. In a related application, the valves described herein may be used to dispense pharmaceuticals and/or ingredients thereof at a pharmacy and/or manufacturing/bottling facility.

In another application, the valves described herein may be used as part of a liquid spice rack. For example, ingredients for recipes may include liquid herbs, spices, and/or flavoring. A user may select a recipe on a mobile app and concoct a blend of ingredients for specific dish, with spices being proportionate to the size of serving. In another embodiment, the valves may be utilized in a smart planter pot in which water and plant food is dispersed via the valves. The dispensed water and/or food may be dispensed in controlled quantities and/or at controlled times, thereby preventing a plant from being over or under nourished. A mobile application may include care instructions for any number of plants. A user may select the appropriate plant and the smart planter may automatically provide the proper nourishment to the plant. In some embodiments, the valves may be utilized in a smart coffee, tea, soda, and/or other beverage machine in which cream, liquid sweeteners, syrups, oils, and/or other additives are dispersed via the valves. The dispensed ingredients may be dispensed in controlled quantities for a personalized “café” to which custom drinks are prepared based on custom creations or pre-selected recipes in the app. In another embodiment, the valves may be utilized in an alcoholic beverage dispenser in which alcohol, bitters, sweeteners, syrups, flavorings, are dispersed via the valves.

In another application, the valves described herein may be used for a virtual reality (VR) olfactory immersion device. Existing VR uses visual and auditory stimulation to create realistic immersive environments. Our sense of smell is our oldest and most powerful sense, and it can be used as a sensory input for further enhancing the VR experience. The valves and/or dispensers described herein may be integrated with VR software to concoct blends related to various environments user is exposed to within a VR experience.

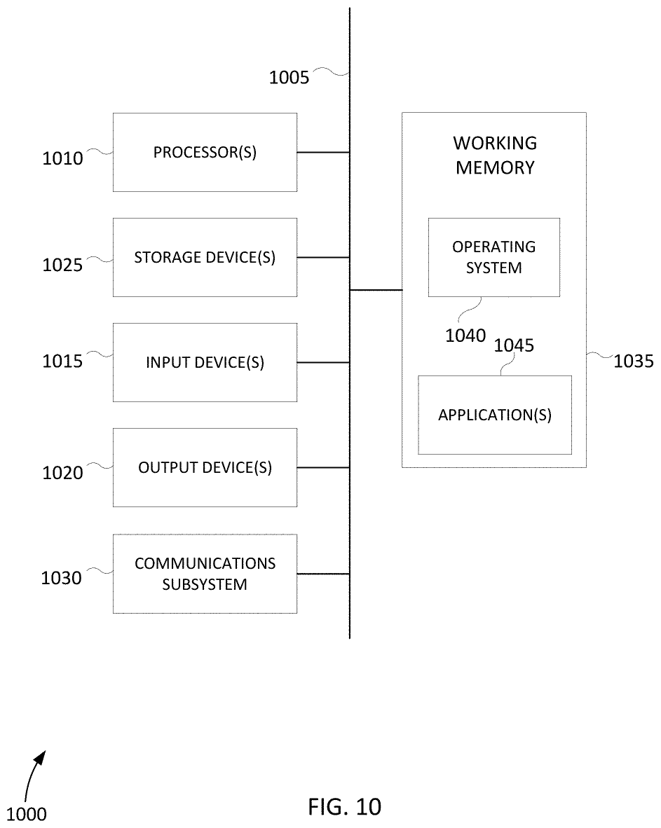

A computer system as illustrated in FIG. 10 may be incorporated as part of the previously described computerized devices. For example, computer system 1000 can represent some of the components of computing devices, such as the controllers for the diffusers, valves, remote devices, and/or other computing devices described herein. FIG. 10 provides a schematic illustration of one embodiment of a computer system 1000 that can perform the methods provided by various other embodiments, as described herein. FIG. 10 is meant only to provide a generalized illustration of various components, any or all of which may be utilized as appropriate. FIG. 10, therefore, broadly illustrates how individual system elements may be implemented in a relatively separated or relatively more integrated manner.

The computer system 1000 is shown comprising hardware elements that can be electrically coupled via a bus 1005 (or may otherwise be in communication, as appropriate). The hardware elements may include a processing unit 1010, including without limitation one or more processors, such as one or more special-purpose processors (such as digital signal processing chips, graphics acceleration processors, and/or the like); one or more input devices 1015, which can include without limitation a keyboard, a touchscreen, receiver, a motion sensor, a camera, a smartcard reader, a contactless media reader, and/or the like; and one or more output devices 1020, which can include without limitation a display device, a speaker, a printer, a writing module, and/or the like.

The computer system 1000 may further include (and/or be in communication with) one or more non-transitory storage devices 1025, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data stores, including without limitation, various file systems, database structures, and/or the like.

The computer system 1000 might also include a communication interface 1030, which can include without limitation a modem, a network card (wireless or wired), an infrared communication device, a wireless communication device and/or chipset (such as a Bluetooth™ device, an 502.10 device, a Wi-Fi device, a WiMAX device, an NFC device, cellular communication facilities, etc.), and/or similar communication interfaces. The communication interface 1030 may permit data to be exchanged with a network (such as the network described below, to name one example), other computer systems, and/or any other devices described herein. In many embodiments, the computer system 1000 will further comprise a non-transitory working memory 1035, which can include a RAM or ROM device, as described above.

The computer system 1000 also can comprise software elements, shown as being currently located within the working memory 1035, including an operating system 1040, device drivers, executable libraries, and/or other code, such as one or more application programs 1045, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such special/specific purpose code and/or instructions can be used to configure and/or adapt a computing device to a special purpose computer that is configured to perform one or more operations in accordance with the described methods.

A set of these instructions and/or code might be stored on a computer-readable storage medium, such as the storage device(s) 1025 described above. In some cases, the storage medium might be incorporated within a computer system, such as computer system 1000. In other embodiments, the storage medium might be separate from a computer system (e.g., a removable medium, such as a compact disc), and/or provided in an installation package, such that the storage medium can be used to program, configure and/or adapt a special purpose computer with the instructions/code stored thereon. These instructions might take the form of executable code, which is executable by the computer system 1000 and/or might take the form of source and/or installable code, which, upon compilation and/or installation on the computer system 1000 (e.g., using any of a variety of available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Moreover, hardware and/or software components that provide certain functionality can comprise a dedicated system (having specialized components) or may be part of a more generic system. For example, a risk management engine configured to provide some or all of the features described herein relating to the risk profiling and/or distribution can comprise hardware and/or software that is specialized (e.g., an application-specific integrated circuit (ASIC), a software method, etc.) or generic (e.g., processing unit 1010, applications 1045, etc.) Further, connection to other computing devices such as network input/output devices may be employed.

Some embodiments may employ a computer system (such as the computer system 1000) to perform methods in accordance with the disclosure. For example, some or all of the procedures of the described methods may be performed by the computer system 1000 in response to processing unit 1010 executing one or more sequences of one or more instructions (which might be incorporated into the operating system 1040 and/or other code, such as an application program 1045) contained in the working memory 1035. Such instructions may be read into the working memory 1035 from another computer-readable medium, such as one or more of the storage device(s) 1025. Merely by way of example, execution of the sequences of instructions contained in the working memory 1035 might cause the processing unit 1010 to perform one or more procedures of the methods described herein.

The terms “machine-readable medium” and “computer-readable medium,” as used herein, refer to any medium that participates in providing data that causes a machine to operate in a specific fashion. In an embodiment implemented using the computer system 1000, various computer-readable media might be involved in providing instructions/code to processing unit 1010 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 1025. Volatile media include, without limitation, dynamic memory, such as the working memory 1035. Transmission media include, without limitation, coaxial cables, copper wire, and fiber optics, including the wires that comprise the bus 1005, as well as the various components of the communication interface 1030 (and/or the media by which the communication interface 1030 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a magnetic medium, optical medium, or any other physical medium with patterns of holes, a RAM, a PROM, EEPROM, EPROM, a FLASH-EPROM, any other memory chip or cartridge, a carrier wave as described hereinafter, or any other medium from which a computer can read instructions and/or code.

The communication interface 1030 (and/or components thereof) generally will receive the signals, and the bus 1005 then might carry the signals (and/or the data, instructions, etc., carried by the signals) to the working memory 1035, from which the processor(s) 1010 retrieves and executes the instructions. The instructions received by the working memory 1035 may optionally be stored on a non-transitory storage device 1025 either before or after execution by the processing unit 1010.

The methods, systems, and devices discussed above are examples. Some embodiments were described as processes depicted as flow diagrams or block diagrams. Although each may describe the operations as a sequential process, many of the operations can be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It should be noted that the systems and devices discussed above are intended merely to be examples. It must be stressed that various embodiments may omit, substitute, or add various procedures or components as appropriate. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known structures and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

The methods, systems, devices, graphs, and tables discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims. Additionally, the techniques discussed herein may provide differing results with different types of context awareness classifiers.

While illustrative and presently preferred embodiments of the disclosed systems, methods, and machine-readable media have been described in detail herein, it is to be understood that the inventive concepts may be otherwise variously embodied and employed, and that the appended claims are intended to be construed to include such variations, except as limited by the prior art.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or +0.1% from the specified value, as such variations are appropriate to in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or +0.1% from the specified value, as such variations are appropriate to in the context of the systems, devices, circuits, methods, and other implementations described herein. As used herein, including in the claims, “and” as used in a list of items prefaced by “at least one of” or “one or more of” indicates that any combination of the listed items may be used. For example, a list of “at least one of A, B, and C” includes any of the combinations A or B or C or AB or AC or BC and/or ABC (i.e., A and B and C). Furthermore, to the extent more than one occurrence or use of the items A, B, or C is possible, multiple uses of A, B, and/or C may form part of the contemplated combinations. For example, a list of “at least one of A, B, and C” may also include AA, AAB, AAA, BB, etc.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

Also, the words “comprise”, “comprising”, “contains”, “containing”, “include”, “including”, and “includes”, when used in this specification and in the following claims, are intended to specify the presence of stated features, integers, components, or steps, but they do not preclude the presence or addition of one or more other features, integers, components, steps, acts, or groups.

What is claimed is:

1. A valve for dispensing metered volumes of fluid, comprising:
a valve body configured to be interfaced with a container holding a fluid, the valve body defining a fluid lumen that extends along at least a portion of a length of the valve body, wherein a valve tip is in communication with the fluid lumen;
an actuator positioned within the valve body;
a plunger positioned within the valve body and in communication with the fluid lumen, the plunger being moveable by the actuator to open and close the fluid lumen, wherein the plunger is biased in a closed direction; and
a controller that is configured to activate the actuator to dispense a volume of fluid from the valve.

2. The valve for dispensing metered volumes of fluid of claim 1, further comprising:
a valve cap.

3. The valve for dispensing metered volumes of fluid of claim 2, wherein:
the controller is disposed in one or both of the valve body or the valve cap.

4. The valve for dispensing metered volumes of fluid of claim 2, wherein:
the valve cap comprises a magnetic element that is used to orient the valve cap when inserted into an external device.

5. The valve for dispensing metered volumes of fluid of claim 2, wherein:
the valve cap funnels fluid from the fluid lumen through a tip of the valve cap.

6. The valve for dispensing metered volumes of fluid of claim 1, wherein:
operation of the actuator is driven by one or both of a volume of fluid to be dispensed and a viscosity of the fluid.

7. The valve for dispensing metered volumes of fluid of claim 1, further comprising:
a fluid level sensor that is configured to detect a presence of the fluid within the container.

8. The valve for dispensing metered volumes of fluid of claim 7, wherein:
the fluid level sensor comprises a capacitive sensor that is coupled with the valve body.

9. The valve for dispensing metered volumes of fluid of claim 7, wherein:
the controller is further configured to determine a total volume of the fluid within the container based on a signal received from the fluid level sensor.

10. A fluid dispensing assembly, comprising:
a container storing a volume of fluid, the container comprising a neck; and
a valve comprising:
a valve body configured to be interfaced with the container, the valve body defining a fluid lumen that extends along at least a portion of a length of the valve body, wherein a valve tip is in communication with the fluid lumen;
an actuator positioned within the valve body;
a plunger positioned within the valve body and in communication with the fluid lumen, the plunger being moveable by the actuator to open and close the fluid lumen, wherein the plunger is biased in a closed direction; and
a controller that is configured to activate the actuator to dispense a metered amount of fluid from the container via the valve.

11. The fluid dispensing assembly of claim 10, wherein:
the plunger is biased in the closed direction by a spring element.

12. The fluid dispensing assembly of claim 10, wherein:
the actuator comprises a solenoid; and
the plunger comprises a ferromagnetic material and is actuated by current passing through the solenoid.

13. The fluid dispensing assembly of claim 10, wherein:
the valve body further defines at least one air return channel.

14. The fluid dispensing assembly of claim 10, wherein:
the fluid lumen is sealed off from electronic components of the valve.

15. The fluid dispensing assembly of claim 10, further comprising:
a Euro dropper disposed between a storage region of the container and the valve.

16. The fluid dispensing assembly of claim 10, wherein:
the valve further comprises an electrical connector disposed on an outer surface of the valve, the electrical connector being coupleable to one or both of a power source and an external processor.

17. The fluid dispensing assembly of claim 10, wherein:
the valve body defines a recess that receives at least a portion of the neck of the container and that secures the valve with the container.

18. The fluid dispensing assembly of claim 10, wherein:
the fluid comprises an essential oil.

19. The fluid dispensing assembly of claim 10, wherein:
the fluid comprises a multivitamin.

20. A method of dispensing a fluid, comprising:
receiving, at a processor of a valve, a signal to dispense a metered volume of a fluid from a container that is interfaced with the valve; and
triggering an actuator of the valve to move a plunger to an open position to dispense the metered volume of the fluid from the container, wherein one or both of a duration and a distance of movement of the plunger to the open position is based at least in part on one or both of an amount of the metered volume and a viscosity of the fluid.
